(19) United States
(12) Patent Application Publication
(10) Pub. No.: US 2022/0016063 A1
(43) Pub. Date: Jan. 20, 2022

(54) COMPOSITIONS THAT ENHANCE IRON ABSORPTION AND METHODS OF USE THEREOF

(71) Applicant: UNIVERSITY OF FLORDIA RESEARCH FOUNDATION, INCORPORATED, Gainesville, FL (US)

(72) Inventors: Sadasivan VIDYASAGAR, Gainesville, FL (US); James COLLINS, Gainesville, FL (US); Regina WOLOSHUN, Gainesville, FL (US)

(21) Appl. No.: 17/311,234

(22) PCT Filed: Dec. 6, 2019
(86) PCT No.: PCT/US2019/065063

§ 371 Date: Jun. 4, 2021

Related U.S. Application Data
(60) Provisional application No. 62/776,645, filed on Dec. 7, 2018.

(51) Int. Cl. A61K 31/198; A61P 7/06

(52) U.S. Cl. CPC A61K 31/198; A61P 7/06

(57) ABSTRACT

Amino acid compositions useful for increasing the amount of the divalent metal-ion transporter 1, DMT1 (encoded by the gene SLC11A2), on the duodenal bmsh border membrane (BBM) are described herein. Methods for increasing the concentration of DMT1 on the duodenal BBM (on trafficking) and increasing iron uptake are also presented. Compositions and methods described herein are useful for treating a disorder or disease associated with iron deficiency in subjects afflicted with such disorders or diseases. Use of these compositions for the treatment of disorders or diseases associated with iron deficiency and in the preparation of a medicament for the treatment of disorders or diseases associated with iron deficiency are also encompassed herein.

### RELATED APPLICATIONS

This application claims priority of U.S. Provisional Application No. 62/776,645 filed Dec. 7, 2018, the entirety of which is incorporated herein by reference for all purposes.

### STATEMENT OF RIGHTS TO INVENTIONS MADE UNDER FEDERALLY SPONSORED RESEARCH

This invention was made with government support under grant Nos. R01 DK074867 and R01 DK109717 awarded by the National Institutes of Health. The government has certain rights in the invention.

### FIELD OF THE INVENTION

Amino acid compositions useful for increasing the amount of the divalent metal-ion transporter 1, DMT1 (encoded by the gene SLC11A2), on the duodenal brush border membrane (BBM) are described herein. Methods for increasing the concentration of DMT1 on the duodenal BBM (on trafficking) and increasing iron uptake are also presented. Compositions and methods described herein are useful for treating a disorder or disease associated with iron deficiency in subjects afflicted with such disorders or diseases. Use of these compositions for the treatment of disorders or diseases associated with iron deficiency and in the preparation of a medicament for the treatment of disorders or diseases associated with iron deficiency are also encompassed herein.

### BACKGROUND OF THE INVENTION

Iron (Fe) is an essential micronutrient that is required for hemoglobin synthesis, energy metabolism, steroid hormone synthesis, DNA synthesis, and cellular protection. The human body contains on average 3-5 grams of Fe. There is no active, regulated excretory mechanism for Fe, and Fe can cause oxidative damage due to its propensity to participate in Fenton chemistry, which results in the production of damaging oxygen radicals. Absorption of dietary Fe must thus be tightly regulated and controlled at the level of the small intestine.

Iron deficiency is the most common micronutrient deficiency worldwide, and approximately 1.6 billion people have anemia, with Fe deficiency as the most common cause.

### SUMMARY

Covered embodiments are defined by the claims, not this summary. This summary is a high-level overview of various aspects and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

Because of the essentiality of Fe for humans, achieving and maintaining a normal Fe status is crucial to improving overall quality of life and preserving health.

In an aspect, a pharmaceutical formulation for use in treating a disease or disorder associated with iron deficiency in a subject in need thereof is presented, wherein the pharmaceutical formulation comprises: a) pharmaceutically active ingredients comprising, consisting essentially of, or consisting of a therapeutically effective amount of each of at least two free amino acids selected from a group of amino acids consisting essentially of aspartic acid, glutamic acid, glutamine, and glycine, wherein the therapeutically effective amount of each of the at least two free amino acids is sufficient to treat the disease or disorder associated with iron deficiency in the subject; and

b) at least one a pharmaceutically inactive ingredient.

In a further aspect, a pharmaceutical formulation for use in treating a disease or disorder associated with iron deficiency is presented, wherein the pharmaceutical formulation comprises:

a) pharmaceutically active ingredients comprising, consisting essentially of, or consisting of a therapeutically effective amount of each of at least two free amino acids selected from a first group of amino acids consisting essentially of aspartic acid, glutamic acid, glutamine, and glycine, wherein the therapeutically effective amount of each of the at least two free amino acids is sufficient to treat the disease or disorder associated with iron deficiency in the subject; b) at least one a pharmaceutically inactive ingredient; and c) optionally further comprising pharmaceutically active ingredients comprising, consisting essentially of, or consisting of a therapeutically effective amount of each of at least one free amino acid selected from a second group of amino acids consisting essentially of cysteine, histidine, and isoleucine,
wherein the therapeutically effective amount of each of the at least one free amino acids is sufficient to treat the disease or disorder associated with iron deficiency in the subject. In a particular embodiment, the second group of amino acids consists essentially of or consists of cysteine or histidine.

In an embodiment of either of these aspects, the pharmaceutical composition does not comprise any one of leucine, lysine, serine, threonine, tryptophan, tyrosine, or valine or any combination thereof. In a further embodiment of either of these aspects, the pharmaceutical composition does not comprise any one of leucine, lysine, serine, threonine, tryptophan, tyrosine, or valine, or any combination thereof.

In an embodiment of either of these aspects and any embodiment thereof, each of the amino acids is an L-amino acid.

In an embodiment of either of these aspects and any embodiment thereof, the pharmaceutical formulation further comprises water as a pharmaceutically inactive ingredient.

In an embodiment of either of these aspects and any embodiment thereof, the at least one pharmaceutically inactive ingredient comprises a pharmaceutically acceptable carrier, buffer, electrolyte, adjuvant, or excipient.

In an embodiment of either of these aspects and any embodiment thereof, the pharmaceutical formulation is sterile.

In an embodiment of either of these aspects and any embodiment thereof, the pharmaceutical formulation is formulated for administration by an enteral, pulmonary, inhalation, intranasal, or sublingual route.

In an embodiment of either of these aspects and any embodiment thereof, the pharmaceutical formulation is for use as a medicament for the treatment of a disease or disorder associated with iron deficiency.

In an embodiment of either of these aspects and any embodiment thereof, the pharmaceutically active ingredients consist essentially of or consist of the therapeutically effective amount of each of the at least two free amino acids selected from the group of amino acids consisting essentially of aspartic acid, glutamic acid, glutamine, and glycine; or the pharmaceutically active ingredients consist essentially of or consist of the therapeutically effective amount of each of the at least two free amino acids selected from the first group of amino acids consisting essentially of aspartic acid, glutamic acid, glutamine, and glycine and, when present, the therapeutically effective amount of each of the at least one free amino acids selected from the second group of amino acids consisting essentially of cysteine, histidine, and isoleucine.

In an embodiment of either of these aspects and any embodiment thereof, the at least two free amino acids consist essentially of or consist of aspartic acid, glutamic acid, glutamine, and glycine.

In an embodiment of either of these aspects and any embodiment thereof, the at least two free amino acids consist essentially of or consist of aspartic acid and glutamic acid.

In an embodiment of either of these aspects and any embodiment thereof, the at least two free amino acids consist essentially of or consist of aspartic acid, glutamic acid, and glutamine.

In an embodiment of either of these aspects and any embodiment thereof, the at least two free amino acids consist essentially of or consist of aspartic acid and glutamine.

In an embodiment of either of these aspects and any embodiment thereof, the at least two free amino acids consist essentially of or consist of aspartic acid, glutamine, and glycine.

In an embodiment of either of these aspects and any embodiment thereof, the at least two free amino acids consist essentially of or consist of aspartic acid and glycine.

In an embodiment of either of these aspects and any embodiment thereof, the at least two free amino acids consist essentially of or consist of glutamic acid and glutamine.

In an embodiment of either of these aspects and any embodiment thereof, the at least two free amino acids consist essentially of or consist of glutamic acid, glutamine, and glycine.

In an embodiment of either of these aspects and any embodiment thereof, the at least two free amino acids consist essentially of or consist of glutamic acid and glycine.

In an embodiment of either of these aspects and any embodiment thereof, the at least two free amino acids consist essentially of or consist of glutamine and glycine.

In an embodiment of either of these aspects and any embodiment thereof, the at least two free amino acids consist essentially of or consist of aspartic acid, glutamic acid, and glycine.

In an embodiment of either of these aspects and any embodiment thereof, a concentration of each of the amino acids present ranges from 0.1 mM to 12 mM or 0.5 mM to 12 mM.

In an embodiment of either of these aspects and any embodiment thereof, when included, a concentration of valine is 10 mM, a concentration of threonine is 8 mM, a concentration of tyrosine is 1.2 mM, a concentration of serine is 10 mM, and a concentration of lysine is 4 mM.

In an embodiment of either of these aspects and any embodiment thereof, the pH ranges from 5.5 to 8.0 or is about 6.5.

In an embodiment of either of these aspects and any embodiment thereof, the disease or disorder associated with iron deficiency comprises iron-deficiency anemia (IDA); anemia associated with chronic kidney disease; iron-refractory, iron-deficiency anemia (IRIDA); anemia associated with inflammation; anemia associated with pregnancy; anemia associated with excessive menstrual blood loss; anemia associated with dietary iron insufficiency; anemia associated with intestinal infections, or anemia associated with inflammatory bowel diseases. In a particular embodiment,

the anemia comprises iron-deficiency anemia (IDA).

In a further aspect, a method for treating a disease or disorder associated with iron deficiency in a subject in need thereof is presented, the method comprising: administering a pharmaceutical composition to the subject in need thereof, wherein the pharmaceutical composition comprises:

a) pharmaceutically active ingredients comprising, consisting essentially of, or consisting of a therapeutically effective amount of each of at least two free amino acids selected from a group of amino acids consisting essentially of aspartic acid, glutamic acid, glutamine, and glycine, wherein the therapeutically effective amount of each of the at least two free amino acids is sufficient to treat the disease or disorder associated with iron deficiency in the subject; and
b) at least one a pharmaceutically inactive ingredient.

In a further aspect, a method for treating a disease or disorder associated with iron deficiency in a subject in need thereof is presented, the method comprising: administering a pharmaceutical composition to the subject in need thereof, wherein the pharmaceutical composition comprises:

a) pharmaceutically active ingredients comprising, consisting essentially of, or consisting of a therapeutically effective amount of each of at least two free amino acids selected from a first group of amino acids consisting essentially of aspartic acid, glutamic acid, glutamine, and glycine, wherein the therapeutically effective amount of each of the at least two free amino acids is sufficient to treat the disease or disorder associated with iron deficiency in the subject; b) at least one a pharmaceutically inactive ingredient; and c) optionally further comprising pharmaceutically active ingredients comprising, consisting essentially of, or consisting of a therapeutically effective amount of each of at least one free amino acid selected from a second group of amino acids consisting essentially of cysteine, histidine, and isoleucine, wherein the therapeutically effective amount of each of the at least one free amino acids is sufficient to treat the disease or disorder associated with iron deficiency in the subject. In a particular embodiment, the second group of amino acids consists essentially of or consists of cysteine or histidine.

In an embodiment of either of these methods, the pharmaceutical composition does not comprise any one of leucine, lysine, serine, threonine, tryptophan, tyrosine, or valine or any combination thereof. In a further embodiment of either of these aspects, the pharmaceutical composition does not comprise any one of leucine, lysine, serine, threonine, tryptophan, tyrosine, or valine, or any combination thereof.

In an embodiment of either of these aspects pertaining to methods, each of the amino acids is an L-amino acid.

In an embodiment of either of these aspects pertaining to methods, the pharmaceutical formulation further comprises water as a pharmaceutically inactive ingredient.

In an embodiment of either of these aspects pertaining to methods, the at least one pharmaceutically inactive ingredient comprises a pharmaceutically acceptable carrier, buffer, electrolyte, adjuvant, or excipient.

In an embodiment of either of these aspects pertaining to methods, the pharmaceutical formulation is sterile.

In an embodiment of either of these aspects pertaining to methods, the pharmaceutical formulation is formulated for administration by an enteral, pulmonary, inhalation, intranasal, or sublingual route.

In an embodiment of either of these aspects pertaining to methods, the pharmaceutically active ingredients consist essentially of or consist of the therapeutically effective amount of each of the at least two free amino acids selected from the group of amino acids consisting essentially of aspartic acid, glutamic acid, glutamine, and glycine; or the pharmaceutically active ingredients consist essentially of or consist of the therapeutically effective amount of each of the at least two free amino acids selected from the first group of amino acids consisting essentially of aspartic acid, glutamic acid, glutamine, and glycine and, when present, the therapeutically effective amount of each of the at least one free amino acids selected from the second group of amino acids consisting essentially of cysteine, histidine, and isoleucine.

In an embodiment of either of these aspects pertaining to methods, the at least two free amino acids consist essentially of or consist of aspartic acid, glutamic acid, glutamine, and glycine.

In an embodiment of either of these aspects pertaining to methods, the at least two free amino acids consist essentially of or consist of aspartic acid and glutamic acid.

In an embodiment of either of these aspects pertaining to methods, the at least two free amino acids consist essentially of or consist of aspartic acid, glutamic acid, and glutamine.

In an embodiment of either of these aspects pertaining to methods, the at least two free amino acids consist essentially of or consist of aspartic acid and glutamine.

In an embodiment of either of these aspects pertaining to methods, the at least two free amino acids consist essentially of or consist of aspartic acid, glutamine, and glycine.

In an embodiment of either of these aspects pertaining to methods, the at least two free amino acids consist essentially of or consist of aspartic acid and glycine.

In an embodiment of either of these aspects pertaining to methods, the at least two free amino acids consist essentially of or consist of glutamic acid and glutamine.

In an embodiment of either of these aspects pertaining to methods, the at least two free amino acids consist essentially of or consist of glutamic acid, glutamine, and glycine.

In an embodiment of either of these aspects pertaining to methods, the at least two free amino acids consist essentially of or consist of glutamic acid and glycine.

In an embodiment of either of these aspects pertaining to methods, the at least two free amino acids consist essentially of or consist of glutamine and glycine.

In an embodiment of either of these aspects pertaining to methods, the at least two free amino acids consist essentially of or consist of aspartic acid, glutamic acid, and glycine.

In an embodiment of either of these aspects pertaining to methods, a concentration of each of the amino acids present ranges from 0.1 mM to 12 mM or 0.5 mM to 12 mM.

In an embodiment of either of these aspects pertaining to methods, when included, a concentration of valine is 10 mM, a concentration of threonine is 8 mM, a concentration of tyrosine is 1.2 mM, a concentration of serine is 10 mM, and a concentration of lysine is 4 mM.

In an embodiment of either of these aspects pertaining to methods, the pH of the pharmaceutical composition ranges from 5.5 to 8.0 or is about 6.5.

In an embodiment of either of these aspects pertaining to methods, the pH is about 6.5.

In an embodiment of either of these aspects pertaining to methods, the disease or disorder associated with iron deficiency comprises iron-deficiency anemia (IDA); anemia associated with chronic kidney disease; iron-refractory, iron-deficiency anemia (IRIDA); anemia associated with inflammation; anemia associated with pregnancy; anemia associated with excessive menstrual blood loss; anemia associated with dietary iron insufficiency; anemia associated with intestinal infections, or anemia associated with inflammatory bowel diseases.

In an embodiment of either of these aspects pertaining to methods, the anemia comprises iron-deficiency anemia (IDA).

In an aspect, a method for treating a subject afflicted with a disease or disorder associated with iron deficiency is presented, the method comprising: administering to the subject afflicted with the disease or disorder associated with iron deficiency a pharmaceutical composition comprising between one and seven selected amino acids and a pharmaceutically acceptable carrier, wherein the selected amino acids consist of aspartic acid, glutamic acid, glutamine, glycine, cysteine, isoleucine, and histidine; and wherein the pharmaceutical composition does not comprise any one of leucine, lysine, serine, threonine, tryptophan, tyrosine, or valine. In a particular embodiment thereof, the pharmaceutical composition does not comprise any one of alanine, arginine, asparagine, or phenylalanine.

In another aspect, a method for treating a subject afflicted with a disease or disorder associated with iron deficiency is presented, the method comprising: administering to the subject afflicted with the disease or disorder associated with iron deficiency a pharmaceutical composition comprising between one and four selected amino acids and a pharmaceutically acceptable carrier, wherein the selected amino acids consist of aspartic acid, glutamic acid, glutamine, and glycine; and wherein the pharmaceutical composition does not comprise any one of leucine, lysine, serine, threonine, tryptophan, tyrosine, or valine. In a particular embodiment thereof, the pharmaceutical composition does not comprise any one of alanine, arginine, asparagine, or phenylalanine.

In a particular embodiment of methods for treating a subject afflicted with a disease or disorder associated with iron deficiency, wherein a concentration of each one of the between one and seven selected amino acids or the between one and four selected amino acids ranges from 6 mM to 10 mM. In a more particular embodiment, when included, a concentration of aspartic acid is 8 mM, a concentration of glutamic acid is 8 mM, a concentration of glutamine is 8 mM, a concentration of glycine is 8 mM, a concentration of cysteine is 8 mM, a concentration of isoleucine is 8 mM, and a concentration of histidine is 8 mM.

In a particular embodiment of methods for treating a subject afflicted with a disease or disorder associated with iron deficiency, the disease or disorder associated with iron deficiency comprises 1) iron-deficiency anemia (IDA), 2) anemia associated with chronic kidney disease; 3) iron-refractory, iron-deficiency anemia (or IRIDA); 4) anemia of inflammation; 5) anemia of pregnancy; 6) anemia associated with excessive menstrual blood loss; and anemia associated with dietary iron insufficiency, or iron deficiency. In a more particular embodiment, the iron deficiency comprises those associated with pregnancy, associated with rapid growth spurts in, for example, adolescents, and associated with severe blood loss due to menstruation.

In yet another aspect, a pharmaceutical composition comprising between one and seven selected amino acids and a pharmaceutically acceptable carrier is described, wherein the selected amino acids consist of aspartic acid, glutamic acid, glutamine, glycine, cysteine, isoleucine, and histidine; and wherein the pharmaceutical composition does not comprise any one of leucine, lysine, serine, threonine, tryptophan, tyrosine, or valine.

In yet another aspect, a pharmaceutical composition comprising between one and four selected amino acids and a pharmaceutically acceptable carrier is described, wherein the selected amino acids consist of aspartic acid, glutamic acid, glutamine, and glycine; and wherein the pharmaceutical composition does not comprise any one of leucine, lysine, serine, threonine, tryptophan, tyrosine, or valine.

In a particular embodiment of the pharmaceutical composition comprising between one and seven selected amino acids or the pharmaceutical composition comprising between one and four selected amino acids, the pharmaceutical composition does not comprise any one of alanine, arginine, asparagine, or phenylalanine. In another particular embodiment of these pharmaceutical compositions, a concentration of each one of the between one and seven selected amino acids or the between one and four selected amino acids ranges from 6 mM to 10 mM. In a more particular embodiment, when included, a concentration of aspartic acid is 8 mM, a concentration of glutamic acid is 8 mM, a concentration of glutamine is 8 mM, a concentration of glycine is 8 mM, a concentration of cysteine is 8 mM, a concentration of isoleucine is 8 mM, and a concentration of histidine is 8 mM. In another particular embodiment, the pharmaceutical compositions are for use in treating a disease or disorder associated with iron deficiency. In a more particular embodiment, the disease or disorder associated with iron deficiency is iron-deficiency anemia (IDA), dietary insufficiency, or iron deficiency. In a more particular embodiment, the iron deficiency comprises those associated with pregnancy, rapid in growth in, for example, adolescents, and severe blood loss due to menstruation. In another particular embodiment, the pharmaceutical compositions are for use in the preparation of a medicament for treating a disease or disorder associated with iron deficiency. In a more particular embodiment, the disease or disorder associated with iron deficiency is iron-deficiency anemia (IDA), dietary insufficiency, or iron deficiency. In a more particular embodiment, the iron deficiency comprises those associated with pregnancy, rapid in growth in, for example, adolescents, and severe blood loss due to menstruation.

All combinations of separately described embodiments are envisaged.

### BRIEF DESCRIPTION OF THE DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIGS. 1A and 1B: Relative DMT1 protein expression from single AA loop studies. FIG. 1A) Histogram depicts relative DMT1 protein expression from single AA loop studies and FIG. 1B) show Western blot images. Duodenal loops were incubated with single AA formulations (pH 6.5) at 37° C. for 45 minutes in a bath of Ringer's buffer. After incubation, luminal liquid was released, and the proximal 5 cm of duodenal mucosa was scraped and BBMVs were isolated. 30 μg protein was loaded per lane and Western blot was performed. Data were quantified and normalized to total proteins on gels (as revealed by Coomassie staining) before normalizing to control. Data are presented as mean±SD; n=1-4 per AA.

FIG. 2: Relative DMT1 protein expression after daily gavage with single AAs for 6 days. Eight- to 10-week-old male Swiss-Webster mice were gavaged with 200 μL of single AA formulations (pH 6.5) daily for 6 days. Mice were sacrificed on day 7, and 5 cm of the proximal duodenal mucosa was scraped for BBMV isolation. Western blots were performed immediately after BBM isolation and blots were quantified and normalized to Coomassie total protein in gels before normalizing to control. Data are presented as mean±SD. The gavage experiment was repeated; n=3-6 per AA.

FIGS. 3A and 3B: Iron-59 flux for control and 4 AA formulations. FIG. 3A and FIG. 3B depict graphical representations of iron-59 flux for control and 4 AA formulations. The 4 AA formulation was created from the AAs that resulted in the greatest DMT1 protein trafficking onto the BBM. FIG. 3A) Flux was significantly higher in the 4 AA group compared to controls. Data were analyzed by nonparametric, unpaired t-test; *p<0.05. B) Conductance is significantly lower after 30 and 60 min in 4 AA compared to control flux studies. Data were analyzed using one-way ANOVA and Sidak's multiple comparison test; *p<0.05 and ****p<0.0001.

FIG. 4A-4C: Iron-59 flux for single AAs from the 4 AA formulation. FIG. 4A) Flux was measured in the presence of single AAs from the 4 AA formulation. FIG. 4B) ⁵⁹Fe flux comparison between 4 AA and 3 AA (excludes Gln) formulations. Flux is not significantly different between 4 AA and 3 AA. FIG. 4C) Conductance during ⁵⁹Fe flux studies in 3 AA and 4 AA formulations. Conductance is significantly lower after one hour in 4 AA vs. 3 AA flux studies. n=16 for 3 AA and n=18 for 4 AA treatment. Data were analyzed using one-way ANOVA and Sidak's multiple comparison test. **p<0.01.

FIG. 5A-5F: DMT1 Western blots from female adult Swiss Webster mice. Mice were housed in wire-mesh cages and given an Fe-deficient diet for FIG. 5A) 12 hours, FIG. 5B) 1 day, FIG. 5C) 2 days, FIG. 5D) 4 days, FIG. 5E) 8 days, and FIG. 5F) 14 days. Upon sacrifice, brush border membrane vesicles (BBMVs) were isolated and DMT1 Western blots were performed. B-actin is shown as a loading control.

FIG. 6A-6C: Non-heme iron levels in various tissues during the time-course study in female adult Swiss Webster mice. Mice were housed in wire-mesh cages and given an Fe-deficient diet for 12 hours, 1, 2, 4, 8, and 14 days. FIG. 6A) Liver, FIG. 6B) kidney, and FIG. 6C) spleen were harvested for non-heme Fe analysis. Significantly lower levels of non-heme Fe were observed in the liver (***p<0.001), kidney (***p<0.001), and spleen (**p<0.01), of the deficient group at 14 days. n=5 per group per time point.

FIG. 7: DMT1 Western blots from male adult Swiss Webster mice. Mice were housed in wire-mesh cages and given an Fe-deficient diet for 12 hours, 1, 2, 4, 8, and 14 days. Upon sacrifice, BBMVs were isolated and DMT1 Western blots were performed. B-actin and/or Coomassie total protein are shown as loading controls.

FIG. 8A-8C: Non-heme iron levels in various tissues during the time-course study in male adult Swiss Webster mice. Mice were housed in wire-mesh cages and given an Fe-deficient diet for 12 hours, 1, 2, 4, 8, and 14 days. FIG. 8A) Liver, FIG. 8B) kidney, and FIG. 8C) spleen were harvested for non-heme Fe analysis. Significantly lower levels of non-heme Fe were observed only in the liver (*p<0.05), of the deficient group at 14 days. Significantly lower levels of non-heme Fe were observed in the spleen at 4 days (*p<0.05) in the deficient group, but this trend was not sustained at 8 or 14 days. n=5 per group per time point.

FIG. 9A-9E: ⁵⁹Fe gavage study in male Swiss Webster mice on chow diet. 2.5 μCi ⁵⁹Fe was administered to each mouse by oral gavage in the presence of control or 4 AA formulations. Mice were sacrificed 2 hours after initial gavage. FIG. 9A) The ⁵⁹Fe absorption (percent of dose absorbed) was significantly higher in the 4 AA group (*p<0.05). FIG. 9B) ⁵⁹Fe activity in blood (*p<0.05) and FIG. 9C) duodenum (*p<0.05) were significantly higher in the 4 AA group. FIG. 9D) ⁵⁹Fe activity in liver was increased but did not achieve statistical significance (p=0.0628). E) ⁵⁹Fe activity in the blood prior to sacrifice was significantly higher at 30 (*p<0.05) and 60 minutes (**p<0.01). n=14 for control and n=15 for 4 AA.

FIG. 10A-10E: ⁵⁹Fe gavage in Swiss Webster male mice given Fe-deficient diet for 10 days prior in wire-mesh cages. 2.5 μCi ⁵⁹Fe was administered to each mouse by oral gavage in the presence of control or 4 AA formulations. The gavage volume was 300 μL. Mice were sacrificed 1 hour after initial gavage to determine FIG. 10A) percent of dose absorbed (*p<0.05), along with FIG. 10B) ⁵⁹Fe activity in blood, FIG. 10C) duodenum, and FIG. 10D) liver (*p<0.05). After the dose was gavaged, blood was taken from the tail at 30 minutes to measure FIG. 10E) ⁵⁹Fe activity (*p<0.05).

FIG. 11A-11B: Repletion data demonstrated by blood hemoglobin. Mice were placed on an iron-deficient diet at weaning for two weeks and baseline Hb was obtained. A daily gavage (200 μL volume) of FeSO₄ occurred after a two hour fast at the following concentrations: 1.575, 3.15, 6.3 mM. A control group was given chow diet and a 0 mM Fe gavage. FIG. 11A) Female Hb data for 14 days of repletion and FIG. 11B) Male Hb data. Data are present as mean±SD.

FIG. 12: Repletion data demonstrated by blood hemoglobin. Mice were placed on Fe-deficient diet for 2 weeks prior in wire overhang cages. Baseline Hb was measured and mice were grouped accordingly so that average baseline Hb was comparable among groups. Baseline Hb ranged from 2.9 to 5 g/dL for males (FIG. 12B) and 3.2 to 7.7 g/dL for females (FIG. 12A).

FIG. 13: Saturation kinetics of repletion data demonstrated by blood hemoglobin for female mice. Mice were placed on an iron-deficient diet at weaning for two weeks and baseline Hb was obtained. A daily gavage (200 μL volume) of 6.3 mM FeSO₄ with and without 4 AA formulation occurred after a two hour fast. Hb was measured every three days thereafter. Female mice achieved half maximal Hb (K_{0.5}) at 16.25 days with control formulation and a Vₘₐₓ of 16.1 g/dL. Female mice achieved half maximal Hb (K_{0.5)} at 10.2 days with 4 AA formulation and a Vₘₐₓ of 14.1 g/dL. Each data point represents the average Hb for each time point within the 21 days.

FIG. 14: Saturation kinetics of repletion data demonstrated by blood hemoglobin for male mice. Mice were placed on an iron-deficient diet at weaning for two weeks and baseline Hb was obtained. A daily gavage (200 μL volume) of 6.3 mM FeSO₄ with and without 4 AA formulation occurred after a two hour fast. Male mice achieved half maximal Hb (K_{0.5)} at 6.6 days with control formulation and a Vₘₐₓ of 10.5 g/dL. Male mice achieved half maximal Hb (K_{0.5}) at 5.01 days with 4 AA formulation and a Vₘₐₓ of 11.8 g/dL. Each data point represents the average Hb for each time point within the 21 days.

FIGS. 15A and 15B: ⁵⁹Fe flux and conductance in male DMT1^{int/int} mice. Duodenal segments from male DMT1 intestine-specific knock-out mice were mounted onto Ussing chamber slides and each half-chamber chamber was bathed in control or 4 AA buffer. Tissues were paired based on conductance, 15 μCi ⁵⁹Fe was added to one side of the chamber, and samples were acquired every 15 minutes from the “cold” side for one hour. Jₙₑₜ was calculated by subtracting Jₛₘ from Jₘₛ. No significant difference was observed with respect to ⁵⁹Fe flux (FIG. 15A) and conductance (FIG. 15B).

FIGS. 16A and 16B: DMT1 Western blots (FIG. 16A) and relative protein expression (FIG. 16B) in loop studies in the presence and absence of Na⁺. Duodenal loops were incubated with control, or 4 AA, or 4 AA no Na⁺ formulations (pH 6.5) at 37° C. for 45 minutes in a bath of Ringer's buffer. After incubation, luminal liquid was released, and the proximal 5 cm of duodenal mucosa was scraped and BBMV were isolated. 30 μg protein was loaded per lane and Western blot was performed. Data were quantified and normalized to Coomassie total protein before normalizing to control. Data from 4 AA and 4 AA no Na⁺ were analyzed by unpaired t-test; n.s.—not significant (p=0.5317). Ctrl n=2, 4 AA n=6, 4 AA no Na⁺ n=5.

FIG. 17: ⁵⁹Fe flux in the presence and absence of Na⁺ in the 4 AA formulation. Duodenal segments from male Swiss-Webster mice were mounted onto Ussing chamber slides and each half-chamber was bathed in 4 AA buffer with or without Na⁺ for 45 min before adding isotope (pH 6.5, 1.5 mM FeSO₄). Tissues were matched based on conductance, 15 μCi ⁵⁹Fe was added to one side of the chamber, and samples were acquired every 15 minutes from the “cold” side for one hour. Jₙₑₜ was calculated by subtracting Jₛₘ from Jₘₛ. n=3 for 4 AA and n=4 for 4 AA no Na⁺. Data were analyzed by unpaired t-test; n.s.—not significant (p=0.301).

FIGS. 18A and 18B: Net ⁵⁹Fe flux in Hamp KO and WT mice. Duodenal segments from male Swiss-Webster mice were mounted onto Ussing chamber slides and each half-chamber was bathed in control or 4 AA buffer. Tissues were paired based on conductance, 15 μCi ⁵⁹Fe was added to one side of the chamber, and samples were acquired every 15 minutes from the “cold” side for one hour. Jₙₑₜ was calculated by subtracting Jₛₘ from Jₘₛ. FIG. 18A) Flux was significantly lower in the KO 5 AA group compared to KO controls. Data were analyzed by nonparametric, unpaired t-test; **p<0.01. FIG. 18B) Conductance is not significantly different at baseline among groups or at 60 min among groups.

FIGS. 19A and 19B: Hemoglobin after 3-week daily gavage period in Hamp KO and WT mice. At weaning, KO and WT mice were administered a daily oral gavage of control, 5 AA, or [3×] 5 AA (except Tyr) formulations after a two hour fast for three weeks. FIG. 19A) No significant changes in Hb were observed in Hamp^{-/-} mice given any of the formulations. FIG. 19B) A significant decrease in Hb was observed in male WT mice administered 5 AA formulation compared to control formulation (**p<0.01).

FIGS. 20A and 20B: Serum ferritin in male (FIG. 20A) and female (FIG. 20B) Hamp KO and WT (FIG. 20C) mice after 3-week daily gavage. Serum ferritin was significantly lower in male KO given 5 AA or 3× 5 AA compared to control formulation. A decreased trend toward significance was observed in female KO given 5 AA compared to control (p=0.1470). No differences in serum ferritin were observed in WT mice.

FIG. 21A-21F: Serum non-heme iron and transferrin saturation in Hamp WT and KO mice after 3-week daily gavage. No significant differences in serum non-heme iron or transferrin-saturation were observed among any of the experimental groups.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. The phrases “effective amount” and “therapeutically effective amount” are used interchangeably. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments. In certain embodiments, an effective amount is an amount that increases the on-trafficking of DMT1 to the plasma membrane of a cell. In certain embodiments, an effective amount is an amount that reduces the symptoms of and/or treats a disease or disorder associated with iron deficiency. In certain embodiments, an effective amount is an amount that reduces the symptoms of and/or treats a disease or disorder associated with iron deficiency.

“Treat,” “treatment”, “treating” and similar terms as used herein in the context of treating a subject refer to providing medical and/or surgical management of a subject. Treatment may include, but is not limited to, administering an agent or composition (e.g., a pharmaceutical composition) to a subject. The term “treatment” or any grammatical variation thereof (e.g., treat, treating, and treatment etc.), as used herein, includes but is not limited to, alleviating a symptom of a disease or condition; and/or reducing, suppressing, inhibiting, lessening, or affecting the progression, severity, and/or scope of a disease or condition.

The effect of treatment may also include reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered, e.g., to reduce the likelihood of recurrence of the disease. A therapeutic agent may be administered prophylactically, i.e., before development of any symptom or manifestation of a disease.

“Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease).

The term “amelioration” or any grammatical variation thereof (e.g., ameliorate, ameliorating, and amelioration, etc.), as used herein, includes, but is not limited to, delaying the onset, or reducing the severity of a disease or condition (e.g., disease or disorder associated with iron deficiency or a complication thereof). Amelioration, as used herein, does not require the complete absence of symptoms.

The terms “condition,” “disease,” and “disorder” are used interchangeably.

All prior patents, publications, and test methods referenced herein are incorporated by reference in their entireties.

### Heme and Non-Heme Iron Absorption

Iron is consumed in the diet as both heme and non-heme Fe. Heme Fe is more bioavailable; up to 30% of heme Fe in food may be absorbed. Non-heme Fe is much less bioavailable. Only about 5% of non-heme Fe in food will be absorbed, because compounds such as phytates, oxalates, and tannins can bind nonheme Fe and reduce its bioavailability. Heme Fe absorption is poorly understood at the mechanistic level. It was once believed to involve heme carrier protein 1 (HCP1), but this transporter was later found to be a folate transporter and renamed proton-coupled folate transporter (PCFT). Non-heme Fe absorption has been widely studied, and much is known about the pathway of its absorption. Absorption primarily occurs in the duodenum and proximal jejunum. First, dietary ferric (Fe³⁺) iron is reduced to ferrous (Fe²⁺) iron by a ferrireductase, duodenal cytochrome B (DCYTB) and by dietary and endogenous factors. Fe²⁺ can then enter the enterocyte via divalent metal-ion transporter 1, DMT1 (encoded by the gene SLC11A2), the intestinal Fe importer. Absorbed Fe may be utilized within enterocytes for metabolic purposes, stored in ferritin, or effluxed by ferroportin, FPN1 (encoded by the gene SLC40A1). Then, Fe is oxidized by the ferroxidase hephaestin, so that Fe³⁺ can bind to transferrin for transport in the blood. Hepcidin, the master Fe regulator, controls Fe efflux into circulation from enterocytes, reticuloendothelial macrophages, and hepatocytes. Hepcidin, which is released in response to high body Fe status and inflammation, binds to FPN1 and causes it to be internalized and degraded, thus limiting Fe efflux into circulation.

### Iron Deficiency and Iron-Deficiency Anemia

Iron deficiency is the most common worldwide micronutrient deficiency. According to the World Health Organization (WHO), the estimated global prevalence of anemia is approximately 1.6 billion people, with Fe deficiency as the most common cause. Because of the essential role of Fe in oxygen delivery to tissues, iron-deficiency anemia (IDA) causes tiredness, fatigue, weakness, and decreased work capacity. Common causes of IDA include dietary insufficiency, and increased Fe demand due to growth, pregnancy, injury, or menstrual blood loss. During pregnancy, IDA is associated with increased risk of miscarriage, low birthweight, premature delivery, and stillbirths. Iron deficiency is defined as decreased total Fe content in the body, and IDA develops once the deficiency is severe enough to impair erythropoiesis. A clinical diagnosis of anemia is a hemoglobin of <12 g/dL for women and <13 g/dL for men. IDA is defined as decreased levels of both transferrin saturation, <16%, and serum ferritin levels, <30 ng/mL. Optimal transferrin saturation is between 25-35% and normal serum ferritin levels are 30-75 ng/mL. Although Fe supplements are commonly utilized, they often contain excessive amounts of Fe that can cause gastrointestinal distress and pain, constipation or diarrhea, nausea, and vomiting. Although there are ongoing supplementation initiatives to correct the global prevalence of anemia, additional research aimed at enhancing intestinal Fe absorption is warranted.

### DMT1: The Intestinal Iron Importer

DMT1 is essential for intestinal Fe absorption and Fe acquisition by erythroid cells and peripheral tissues. It requires a proton for the transport of a divalent Fe ion. DMT1 is not specific for Fe; as its name implies, it can also transport other divalent cations such as Mn²⁺ or Cd²⁺, and possibly Cu²⁺. DMT1 is an integral membrane protein with 12 transmembrane domains and is located on the apical side of duodenal enterocytes. DMT1 is expressed as a 90-100 kDa protein, which is higher than the predicted mass based on the AA sequence (62 kDa). This is due to significant glycosylation; at least 40% of its molecular weight is attributed to glycosylation.

Because Fe absorption must be tightly controlled, DMT1 is regulated in several ways including at the level of transcription, post-transcription, and post-translation. At the transcriptional level, the trans-acting factor hypoxia-inducible factor 2α (HIF2α) is expressed during Fe deficiency, which transactivates intestinal SLC11A2 expression. Another level of regulation involves iron-responsive element/iron-regulatory protein (IRE/IRP) interactions. Interestingly, four DMT1 isoforms are created by alternative splicing, forming either the DMT1A+, DMT1A−, DMT1B+, or DMT1B− forms. DMT1A+ is expressed in the duodenum and contains an IRE. (DMT1B isoforms are expressed in blood cell lines.) When intracellular Fe levels are low, the IRPs can bind to the IRE in the 3′ untranslated region of the DMT1 mRNA transcript, which stabilizes the transcript, thus allowing an increase in protein translation. Conversely, when cellular Fe levels are sufficient, IRPs will not bind to the IRE, and stability of the transcript will decrease, lowering DMT1 protein levels. Lastly, post-translational modifications of DMT1 involve its glycosylation and trafficking from intracellular compartments to the apical membrane and vice versa. Moreover, in Caco-2 cells given Fe, DMT1 was endocytosed rapidly and was detected in the apical cytoplasm above the nucleus. Within 10 minutes of Fe exposure, more than 30% of DMT1 was internalized. This trend was also observed in the Belgrade rat, which possesses a missense mutation which causes the AA conversion, G185R, in SLC11A2 (b/b) which causes severe systemic Fe deficiency. When Belgrade rats and phenotypically normal littermates (+/b) were given a bolus of Fe, DMT1 was internalized into cytoplasmic vesicles 1.5 hours later. Also, in Wistar rats fed an Fe-deficient diet for 8-10 weeks, immunohistochemical localization of DMT1 showed high expression on the apical membrane of duodenal enterocytes. These observations highlight the importance of elucidating the proper dose of Fe in supplements, as supplements with too much Fe could have an inhibitory effect on DMT1 trafficking and thus Fe absorption.

Research described herein aims to create and test AA formulations that can improve Fe status in rodent models of IDA.

Currently available Fe supplements can irritate the mucosal lining, causing intestinal pain and discomfort along with constipation. Untoward impacts of excessive Fe on the colonic microflora are also a possible negative side effect of current Fe supplementation regimens. It is thus crucial to develop a formulation that can enhance Fe absorption without causing such adverse effects. The approach described herein was to lower the amount of Fe needed in a supplement by administering in conjunction with select AAs and/or formulations of select AAs. Introducing AAs into the intestinal lumen can facilitate trafficking of DMT1 to the BBM, potentially enhancing Fe absorption, and possibly serving as a better treatment for IDA.

Testing AA formulations in murine models allows for basic characterization of their effects in advance of translational work to be performed with human duodenal samples. Studies described herein characterize Fe flux and to test AA formulations for trafficking of DMT1 onto the BBM and such information is predictive of response in human tissues. Accordingly, formulations described herein are well applied to objectives directed to improving Fe status in humans suffering with Fe-related disorders.

AA formulations are described herein for use in treating Fe-related disorders, as are methods for treating Fe-related disorders wherein AA formulations are administered to a subject in need thereof Also encompassed herein are AA formulations for use in the preparation of medicaments for treating Fe-related disorders.

In an embodiment, a method for treating a subject afflicted with a disease or disorder associated with iron deficiency, the method comprising: administering to the subject afflicted with the disease or disorder associated with iron deficiency a pharmaceutical composition comprising between one and four selected amino acids and a pharmaceutically acceptable carrier, wherein the selected amino acids consist of aspartic acid, glutamic acid, glutamine, and glycine; and wherein the pharmaceutical composition does not comprise any one of leucine, lysine, serine, threonine, tryptophan, tyrosine, or valine. In a more particular embodiment, the pharmaceutical composition comprises seven selected amino acids and a pharmaceutically acceptable carrier, wherein the selected amino acids consist of aspartic acid, glutamic acid, glutamine, glycine, cysteine, isoleucine, and histidine. In another particular embodiment, the pharmaceutical composition does not comprise any one of alanine, arginine, asparagine, leucine, lysine, serine, threonine, tryptophan, tyrosine, or valine. In a still more particular embodiment, the pharmaceutical composition comprises seven selected amino acids and a pharmaceutically acceptable carrier, wherein the selected amino acids consist of aspartic acid, glutamic acid, glutamine, glycine, cysteine, isoleucine, and histidine and the pharmaceutical composition does not comprise any one of alanine, arginine, asparagine, leucine, lysine, serine, threonine, tryptophan, tyrosine, or valine. Further to the above, the method comprises administering any and all combinations of the above selected amino acids consisting of aspartic acid, glutamic acid, glutamine, cysteine, isoleucine, histidine, and glycine. The method also comprises administering any and all combinations of the above selected amino acids consisting of aspartic acid, glutamic acid, glutamine, and glycine.

A formula for determining the number of different combinations encompassed thereby is 2ⁿ−1, wherein n equals the number of different amino acids in a select list of amino acids (e.g., 4 amino acids). The total number of different combinations of aspartic acid, glutamic acid, glutamine, and glycine is, therefore, 15 different combinations (2⁴−1). For the sake of simplicity, each of the select amino acids is referred to with the standard single capital letters for amino acids as follows: aspartic acid (D), glutamic acid (E), glutamine (Q), and glycine (G). The different combinations are as follows: D, E, Q, G, DE, DQ, DG, EQ, EG, QG, DEQ, DEG, DQG, EQG, and DEQG. The formula applies to pharmaceutical compositions comprising the select four amino acids and uses thereof for treating Fe-related disorders and for preparing medicaments for treating Fe-related disorders (a disease or disorder associated with iron deficiency).

The above formula and reasoning are equally applied to any combinations of select amino acids described herein.

The term “consisting essentially of” as used herein, limits the scope of the ingredients and steps to the specified materials or steps and those that do not materially affect the basic and novel characteristic(s) of the present invention, e.g., compositions and use thereof for the treatment of Fe-related disorders and methods for treating Fe-related disorders. For instance, by using “consisting essentially of” the therapeutic composition does not contain any unspecified ingredients including, but not limited to, free amino acids, di-, oligo-, or polypeptides or proteins; and mono-, di-, oligo-, polysaccharides, and carbohydrates that have a direct beneficial or adverse therapeutic effect on treatment of Fe-related disorders. Also, by using the term “consisting essentially of” the composition may comprise substances that do not have therapeutic effects on the treatment of Fe-related disorders; such ingredients include carriers, excipients, adjuvants, flavoring agents, etc. that do not affect the health or function of the intestinal epithelium.

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

The disclosure described herein may be practiced in the absence of any element or elements, limitation or limitations, which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

### EXAMPLES

Example 1 overview: Formulate and test an AA combination that causes DMT1 to traffic onto the duodenal BBM, thus potentially increasing Fe flux. Approach: Ligated loop studies were conducted on 8-10-week-old male Swiss-Webster mice. Duodenal segments were isolated and filled with single AA formulations and incubated in oxygenated buffer. BBM proteins were then isolated by a standard procedure for Western blot analysis to assess DMT1 protein levels. Swiss-Webster mice were also gavaged daily for 6 days with single AA solutions. The mice were sacrificed on day 7 and the proximal 5 cm of the duodenal mucosa was isolated and processed as described herein for analysis of DMT1 protein expression. A formulation has been created with the four AAs that resulted in the greatest BBM expression of DMT1 relative to controls. This formulation has been tested in Ussing chamber ⁵⁹Fe flux studies. It has also been tested in Fe-deficient mice in a depletion/repletion experiments. Michaelis-Menten kinetic experiments (⁵⁹Fe flux versus AA concentration) may be performed to modify and adjust the formulation as necessary, to potentially enhance Fe absorption further.

Example 2 overview: Formulate an AA combination that causes DMT1 to traffic off the duodenal BBM and test this formulation in murine models of HH. Approach: Ligated loop and in vivo gavage studies in Swiss-Webster mice were used to identify AAs that cause DMT1 to traffic off the BBM. The AA formulation has been tested in Ussing chamber flux experiments and in Hamp KO mice and rats, which are models of early onset (i.e. juvenile) HH in humans. Animals have been gavaged daily with the AA formulation at pre-weaning (19 days old) for 14-21 days. Physiological studies have been performed and biomarkers of Fe status assessed to determine if the AA formulation blunted (or prevented) Fe loading.

Example 3 overview: Characterize Fe flux (heme and non-heme) in human intestinal samples and test the AA formulations in these samples. Approach: Human duodenal samples will be resected from Fe-deficient patients undergoing the Whipple procedure and from normal organ donors. Samples will be freshly transported and prepared for Ussing chamber ⁵⁹Fe flux studies. The flux results will be used to characterize general Fe kinetics in human samples, and the formulations from Examples 1 and 2 will also be tested to determine how absorption is altered in human tissue samples. ⁵⁹Fe-labeled heme will also be used to assess how different AA formulations influence heme Fe absorption. In addition, the present inventors have recently shown that Sprague-Dawley rats can absorb heme Fe, so Hamp KO rats have been incorporated into these studies to increase translational potential.

Significance of Proposal: Incorporation of AAs into drinks or supplements is envisioned to translate into treating, for example, IDA. We have shown that certain AAs can cause DMT1 to traffic onto the BBM, which enhanced Fe flux, serving to potentially treat IDA more effectively. Translating to human intestinal samples will provide further insight into potential treatments for Fe-deficiency.

### Example 1

### Select Amino Acids Increase DMT1 Expression on the BBM and Subsequently Increase Iron Absorption

### Background

A screen of the 20 amino acids was performed to determine the influence of DMT1 trafficking onto the BBM. Four amino acids that caused the greatest amount of DMT1 on the BBM were selected and made into a formulation and tested by Ussing chamber flux studies, in vivo depletion/repletion studies and ⁵⁹Fe gavage studies. The AA formulation has translational potential to treat iron-deficiency anemia, by bringing more DMT1 to the BBM, which may allow for a smaller dose of Fe to be administered in a supplement.

### Methods

### Loop Studies

Blind loop studies were conducted on 8-10-week-old male Swiss-Webster mice, as they are a commonly used outbred strain. Males were selected for initial screening purposes, but experiments will be repeated in female mice. Mice were sacrificed and duodenal segments (approximately 12 cm) were isolated and flushed with Ringer's buffer. One end of the duodenum was tied off, and 300 μL of control or single AA formulations (pH 6.5, 294-305 mOsm) were added into the lumen. The opposite end was then tied off and the loop was incubated and bubbled with 95% O₂ and 5% CO₂ for 45 minutes in a bath of Ringer's buffer (pH 7.4, 296 mOsm, 37° C.). After incubation, the luminal liquid was released. The mucosa of the proximal 5 cm of intestine (just beyond the ligament of Treitz) was scraped into 9.9 mL lysis buffer (300 mM mannitol, 5 mM EGTA, 12 mM Tris-HCl, pH 7.1) with 0.1 mL Halt Protease Inhibitor Cocktail, EDTA-free 10× (Thermo Scientific, catalog #78439). A standard protocol for isolating BBM protein was performed for Western blot analysis (described below).

### Western Blot Analysis

Isolation of Brush-Border Membrane Vesicles (BBMV): A light scrape of the duodenal mucosal was placed in 9.9 mL ice-cold lysis buffer with 0.1 mL Halt Protease Inhibitor Cocktail, EDTA-free 100×. Samples were homogenized on ice using an IKA T25 Ultra Turrax device for 2 min. Following homogenization, 500 μL 1 M MgCl2 was added and the sample was rotated at 4° C. for 10 minutes. Samples were then centrifuged at 10,000 rpm for 25 min using a JA-12 rotor in a Beckman-Coulter ultracentrifuge. The supernatant was transferred to new round-bottom tubes and centrifuged again at 19,000 rpm for 40 minutes using a JA-17 rotor. The remaining pellet after the second centrifugation step contained the BBMVs. The pellet was dissolved in 50 μL Ringer's buffer with protease inhibitor at a final concentration of 1×. Protein levels were quantified using Pierce BCA protein assay kit (Thermo Scientific, catalog #23225) using albumin protein standards.

Sodium Dodecyl Sulfate-Polyacrylamide Gel Electrophoresis (SDS-PAGE): Protein samples, each containing 30 μg protein, were mixed with 4× Laemmli buffer and 1 M dithiolthreitol (ratio of buffer to DTT was 20:1) and incubated at 37° C. for 30 minutes. 30 μg of protein was then loaded into each lane of 8% polyacrylamide gels. SDS-PAGE was run at 90 V for 30 minutes and increased to 120 V for the duration of the electrophoresis.

Western Blot: Proteins in gels were transferred to PVDF membranes at 60 V for two hours. PVDF membranes were blocked in Odyssey blocking buffer (Licor, catalog #927-50000), diluted 1:2 with TBS, for one hour at room temperature under gentle agitation. Membranes were then incubated with DMT1 primary antibody (courtesy of François Canonne-Hergaux, French Institute of Health and Medical Research, Bordeaux, France) at a 1:2000 dilution in Odyssey blocking buffer at 4° C. for at least 16 hours. Blots were rinsed with TBST for 15 minutes under fast agitation for a total of three washes. Blots were then incubated with IRDye 800CW donkey anti-rabbit secondary antibody (Licor, catalog #925-32213) diluted 1:10000 in Odyssey blocking buffer for one hour at room temperature. Blots were again rinsed with TBST for 15 minutes under fast agitation for a total of three washes. Blots were subsequently imaged and protein band signals were quantified with a Licor Odyssey CLx immunofluorescent instrument. Protein band intensities were then normalized to total protein of each lane stained with Coomassie (Gel Analyzer software) and all data was normalized to respective control lanes on the same blot.

### Single AA Gavage Studies

Eight- to ten-week-old male Swiss-Webster mice were gavaged daily in the morning for 6 days with 200 μL of control or single AA formulations (pH 6.5, 294-305 mOsm). The mice were sacrificed on day 7 and the proximal 5 cm of the duodenum was isolated, and the mucosa was immediately scraped into lysis buffer containing protease inhibitor (pH 7.1). Similar to the loop study, BBM isolation and Western blots were immediately performed, and all data were quantified and normalized to controls.

### Formulation Design

A formulation was created which included the four AAs that resulted in the greatest expression of DMT1 relative to the control from loop studies which included: glutamine, aspartic acid, glutamic acid, and glycine (Table 4). The salts, pH of 6.5, and buffering capacity of this solution have been optimized for Fe flux studies by the present inventors (Table 3). This formulation was utilized for the studies presented in this research, as described below.

### Ussing Chamber Flux Studies

Ussing chamber technology was utilized to measure Fe flux in the presence of AA formulations. The Ussing chamber was balanced for at least 30 minutes prior to mounting duodenal mouse tissues. Tissues were bathed bilaterally in 10 mL of control or AA-containing buffers, containing 1.5 mM FeSO₄.7H₂O (“cold” Fe), and were bubbled with a 95% O₂ and 5% CO₂ gas mixture. Experiments were performed under conditions in which the voltage was clamped to zero, so that there was no net passive diffusion of ions and no driving force for paracellular flux. Duodenal epithelial organ cultures were mounted and equilibrated for approximately 10 minutes and were paired for mucosal-to-serosal and serosal-to-mucosal flux based on similar conductance values. Blank measurements (500 μL) were collected from the “cold” (non-radioactive) side. 15 μCi of ⁵⁹Fe (PerkinElmer, catalog #NEZ037500UC) neutralized with N-methyl-D-glucamine (NMDG) was added to each chamber, on either the mucosal or serosal side, depending upon tissue pairing. At 15-minute intervals, 500 μL samples were collected from the “cold” side for 60 minutes, and 500 μL buffer were added back to maintain a fixed volume. At the end of each experiment, 100 μL of “hot” radioactive Fe from each chamber were collected. Radioactivity in the samples was measured using a PerkinElmer gamma counter, and net Fe flux was calculated as Jₙₑₜ=Jₘₛ−Jₛₘ, where Jₘₛ is mucosal to serosal flux (absorption) and Jₛₘ is serosal to mucosal flux (secretion).

Effects of Sodium on DMT1 Trafficking and ⁵⁹Fe Flux: To determine if sodium (Na⁺) influences trafficking of DMT1 and Fe flux, loop and flux studies were performed with the 4 AA formulation in the presence and absence of Na⁺, replacing all compounds containing Na⁺ with NMDG and Hepes (formulations can be found in the Tables below. Formulations without both Na⁺ and AAs were also tested. Here, duodenal epithelial organ cultures were incubated with the respective buffer for 45 minutes before addition of isotope; however, future studies will not use this longer incubation time since tissue viability became an issue.

### DMT1 Induction Study

Eight-week-old male and female Swiss Webster mice were placed on the control-iron diet (50 ppm Fe) for 5 days prior to initiating experimentation. The experimental group was given a low-iron diet (3-5 ppm Fe, Engivo) and was housed in overhanging wire mesh cages. The control group was given a control diet (50 ppm Fe) throughout and housed in conventional static cages. The time points for sacrifice were as follow: 0.5, 1, 2, 4, 8, and 14 days. Brush-border membrane vesicles were isolated and Western blots were performed for DMT1 protein quantification. Liver, kidney, and spleen non-heme iron was also measured using a standard colorimetric assay.

### Iron Depletion-Repletion Study

To establish the amount of iron needed to steadily replete a mouse, the concentration was determined theoretically to be 3.15 mM in a 200 μL gavage dose, based on daily food intake and iron requirement of a rodent (35 mg/kg Fe). Two additional concentrations were included in this study: 1.575 mM and 6.3 mM, which are half and double the theoretical concentration, respectively. A control group was given 0 mM Fe gavage on chow diet to obtain normal Hb values. The Fe was dissolved in control formulation (Table 3). Three-week-old male and female Swiss-Webster mice were placed on an Fe-deficient diet in wire-overhang cages for two weeks. After two weeks baseline hemoglobin (Hb) was measured. Daily gavage began in the evening for 14 days. An additional trial included FeSO₄ and ferrous fumarate at 6.3 mM and 12.6 mM per iron source, along with a control group with 0 mM Fe gavage on control diet (50 ppm Fe).

Three-week-old male and female Swiss-Webster mice were placed on an Fe-deficient diet for two weeks. After two weeks baseline hemoglobin (Hb) was measured and mice were randomized into groups so that average starting Hb values are similar. They were gavaged every evening after a 1.5 hour fast (just before the “active” phase) with a 200 μL volume containing 6.3 mM FeSO₄ in control or 4 AA formulations. Food was given back 30 minutes after daily gavage. Hemoglobin was measured every three days to track repletion. They remained on the Fe-deficient diet throughout the study. This study was performed twice: one trial at pH 6.5 and the second trial at pH 3.5.

### Short-Term ⁵⁹Fe Gavage Study

Eight- to ten-week-old male Swiss Webster mice were maintained on chow diet until the experiment. A two hour fast occurred before 2.5 μCi ⁵⁹Fe was administered to each mouse by oral gavage in the presence of control or 4 AA formulations. The gavage volume was 300 μL. After the dose was gavaged, blood was taken from the tail at 30 and 60 minutes to measure ⁵⁹Fe activity. Mice were sacrificed 2 hours after initial gavage to determine percent of dose absorbed, along with ⁵⁹Fe activity in blood, duodenum, and liver. A similar experimental approach was performed on Swiss Webster male mice that were given an Fe-deficient diet for 10 days prior to gavage. One experiment was performed as described above and another group was sacrificed after 60 minutes, taking blood from the tail at 30 minutes.

### Iron Flux Studies in DMT1^{int/int} Mice

Male and female adult DMT1^{int/int} (129S6 background) were utilized in this study. The flux studies were performed with control and 4 AA formulations. Because intestinal DMT1 is absent in this KO model, these studies will facilitate an assessment of the degree of specificity of formulations described herein for DMT1.

### Statistical Analysis

Unpaired t-test or one- and two-way ANOVA with Tukey's or Sidak's post hoc tests for multiple comparisons were utilized using GraphPad Prism 7 and Origin. Quantified Western blot data are displayed as mean±SD.

### Results

### Loop and Gavage Studies

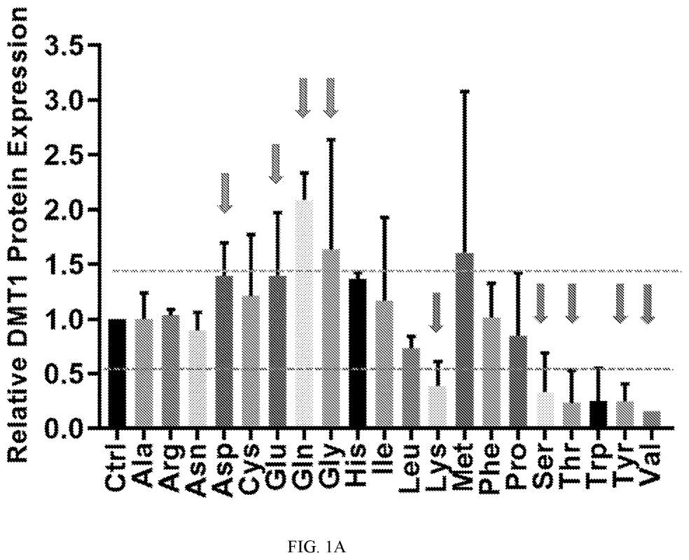

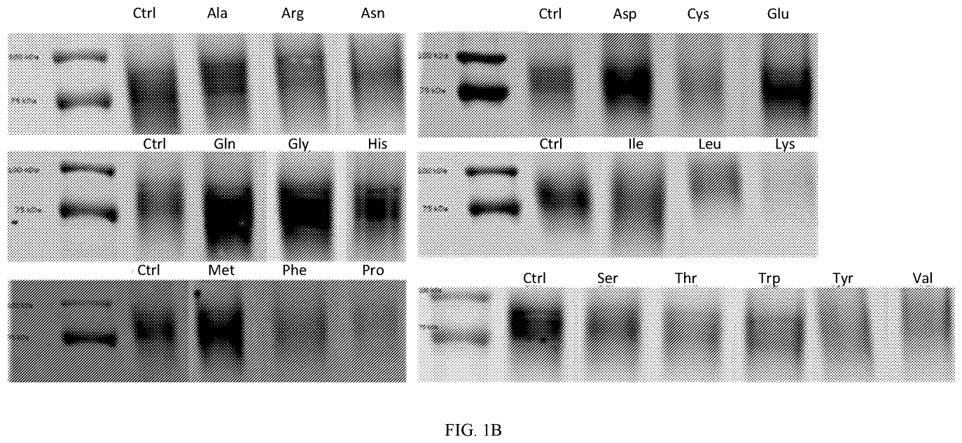

After completion of pilot studies and observing an effect of AAs on DMT1 trafficking off the BBM, the next necessary experiments were to determine which AAs promote on or off trafficking. This was accomplished by performing both ex vivo loop studies and in vivo gavage studies. FIG. 1 displays relative DMT1 protein expression for each AA compared to control (expression set to 1) in loop experiments. A relative protein expression threshold of 1.4 or higher was selected and the AAs that increased DMT1 expression beyond this threshold were selected for the formulation for on trafficking described herein. These include aspartic acid, glutamic acid, glutamine, and glycine. Methionine was excluded, as it has been shown to have carcinogenic properties.

TABLE 1
Formulation for Single Amino Acid Loop and Gavage Studies

| | Molecular weight | Concentration |
|---|---|---|
| Compound | (g/mol) | (mM) |
| NaCl | 58.44 | 105 |
| KCl | 74.55 | 5.2 |
| NaH₂PO₄ | 120 | 7.6 |
| Na₂HPO₄ | 142 | 1.4 |
| MgCl₂ | 95.22 | 1.2 |
| CaCl₂•2H₂O | 147.03 | 1.2 |
| Na Citrate Monobasic | 214.11 | 25 |
| Single amino acid | variable | variable |

Formulations were adjusted to pH 6.5 and osmolarity was measured pH 6.5, 297 mOsm (before AA addition).

TABLE 2
Amino Acid Concentrations for Loop and Gavage Studies

| Amino Acid | Concentration (mM) |
|---|---|
| Alanine | 8 |
| Arginine | 8 |
| Asparagine | 8 |
| Aspartic acid | 8 |
| Cysteine | 8 |
| Glutamate | 8 |
| Glutamine | 8 |
| Glycine | 8 |
| Histidine | 8 |
| Isoleucine | 8 |
| Leucine | 8 |
| Lysine | 4 |
| Methionine | 8 |
| Phenylalanine | 8 |
| Proline | 8 |
| Serine | 10 |
| Threonine | 8 |
| Tryptophan | 8 |
| Tyrosine | 1.2 |
| Valine | 10 |

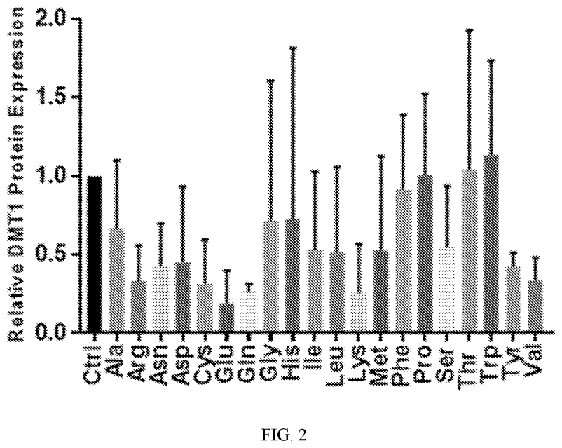

Gavage results (FIG. 2) inherently have variation and do not support the loop data regarding the AAs that led to increased DMT1 BBM trafficking. The results demonstrate that the AAs may not have a chronic effect when gavaged once per day for six days in the morning, especially because this gavage occurs during the inactive phase of mice, when they are not normally eating. Additional experiments include, for example, gavaging Fe and AAs in the evening to model a more physiological setting. The DMT1 BBM off-trafficking results will be discussed further in Example 2 below.

### Ussing Chamber Flux Studies

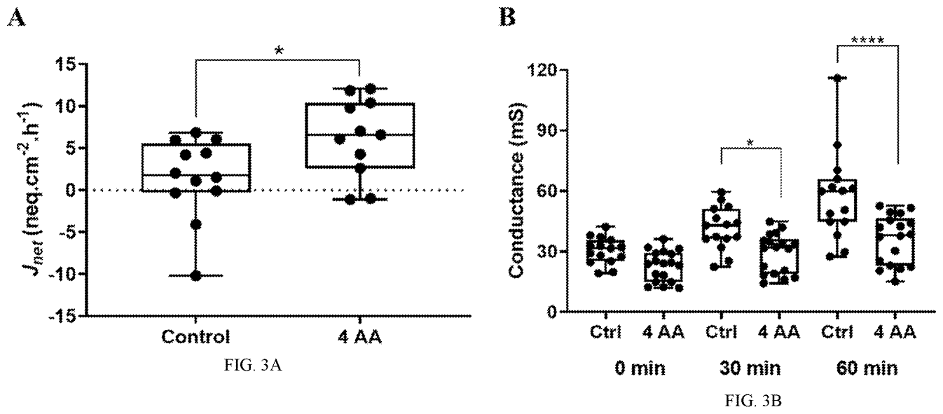

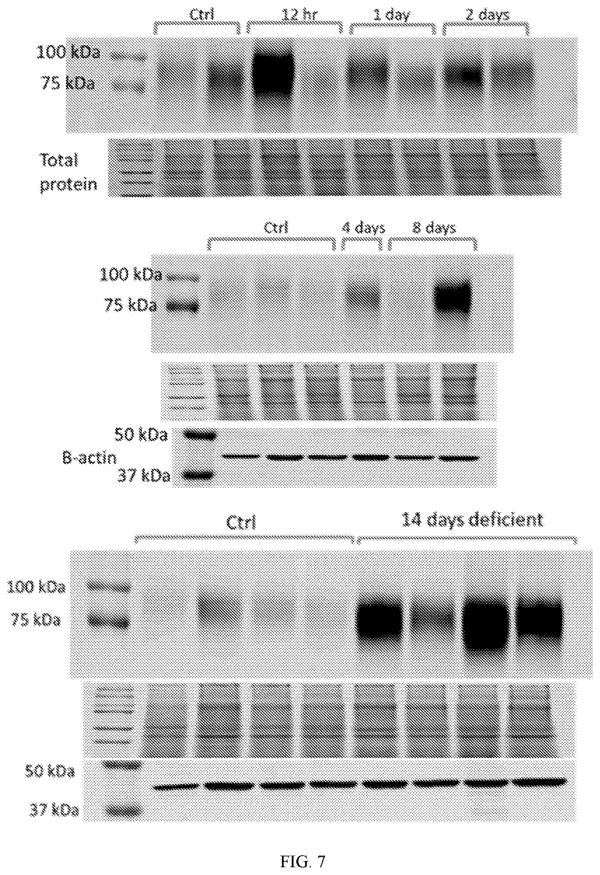

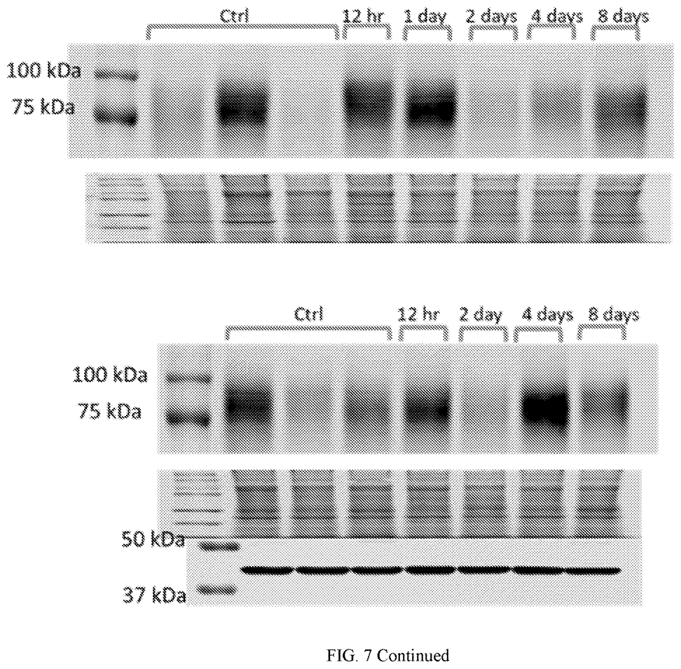

Following loop and gavage studies, the 4 AA formulation created based off loop results was tested in the Ussing chamber, to assess Fe transport in duodenal epithelial organ cultures. ⁵⁹Fe flux studies demonstrated a significant increase in flux (p<0.05) in the 4 AA group (n=11) compared to control (n=12), as shown in FIG. 3. Conductance was significantly increased in control tissues compared to 4 AA-exposed tissues at 30 minutes (p<0.05) and 60 minutes (p<0.0001), demonstrating a protective effect of the 4 AA with more intestinal integrity (FIG. 7). Note that a higher conductance value represents a leakier tissue.

TABLE 3
Control Formulation for Flux Studies

| | Molecular weight | Concentration |
|---|---|---|
| Compound | (g/mol) | (mM) |
| NaCl | 58.44 | 103 |
| KCl | 74.55 | 5.2 |
| NaH₂PO₄ | 120 | 7.6 |
| Na₂HPO₄ | 142 | 1.4 |
| MgCl₂ | 95.22 | 1.2 |
| CaCl₂•2 H₂O | 147.03 | 1.2 |
| Na Citrate Monobasic | 214.11 | 25 |
| FeSO₄•7 H₂O | 278.01 | 1.5 |

Formulations were adjusted to pH 6.5 and osmolarity was measured at 296 mOsm.
Note:
FeSO₄•7 H₂O is only present in Ussing chamber flux studies.

TABLE 4
On-trafficking 4 AA Formulation

| | Molecular weight | Concentration |
|---|---|---|
| Compound | (g/mol) | (mM) |
| NaCl | 58.44 | 86 |
| KCl | 74.55 | 5.2 |
| NaH₂PO₄ | 120 | 7.6 |
| Na₂HPO₄ | 142 | 1.4 |
| MgCl₂ | 95.22 | 1.2 |
| CaCl₂•2 H₂O | 147.03 | 1.2 |
| Na Citrate Monobasic | 214.11 | 25 |
| FeSO₄•7 H₂O | 278.01 | 1.5 |
| Glutamine | 146.145 | 8 |
| Aspartic acid | 133.103 | 8 |
| Glutamic acid | 187.130 | 8 |
| Glycine | 75.067 | 8 |

Formulations were adjusted to pH 6.5 and osmolarity was measured at 294 mOsm.
Note:
concentration of FeSO₄•7 H₂O is only relevant in Ussing chamber flux studies (subsequent studies involving 4 AA formulation indicate the concentration of FeSO₄•7 H₂O used).

Formulations were adjusted to pH 6.5 and osmolarity was measured at 294 mOsm. Note: concentration of FeSO₄.7 H₂O is only relevant in Ussing chamber flux studies (subsequent studies involving 4 AA formulation indicate the concentration of FeSO₄.7 H₂O used).

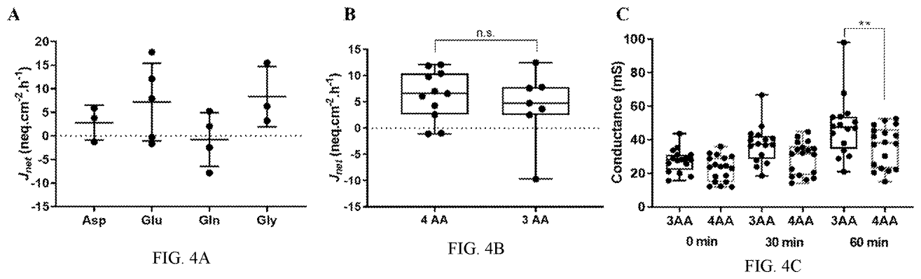

To further refine the 4 AA formulation, flux studies with individual AAs from the 4 AA formulation were performed to determine the effect of each AA on net Fe flux (FIG. 4A). Glutamine resulted in the lowest flux compared to the other AAs, so a formulation was created that excluded glutamine. Flux studies were again performed, and there was no significant difference between net flux of 3 AA and 4 AA (FIG. 4B). Conductance values during the 4 AA experiments were significantly less than the 3 AA experiments after one hour, demonstrating the 4 AA formulation is superior in maintaining the intestinal integrity (FIG. 4C). Therefore, the 4 AA formulation was utilized and tested in the subsequent experiments.

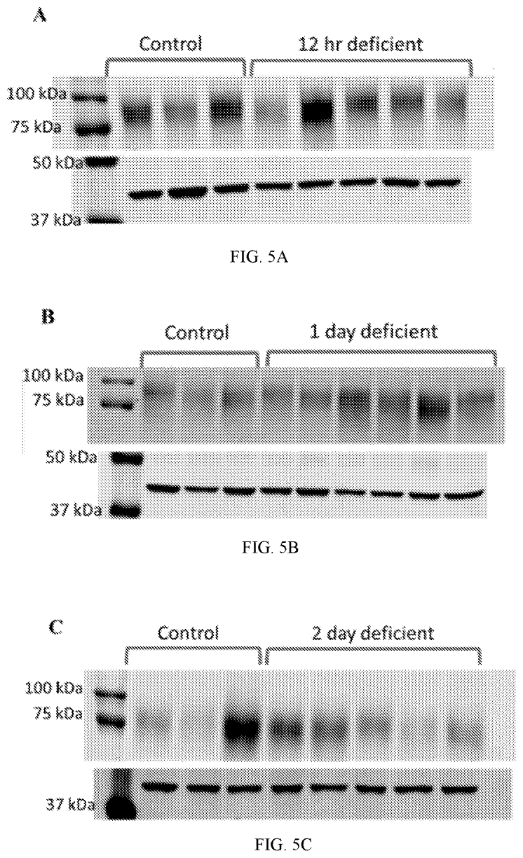

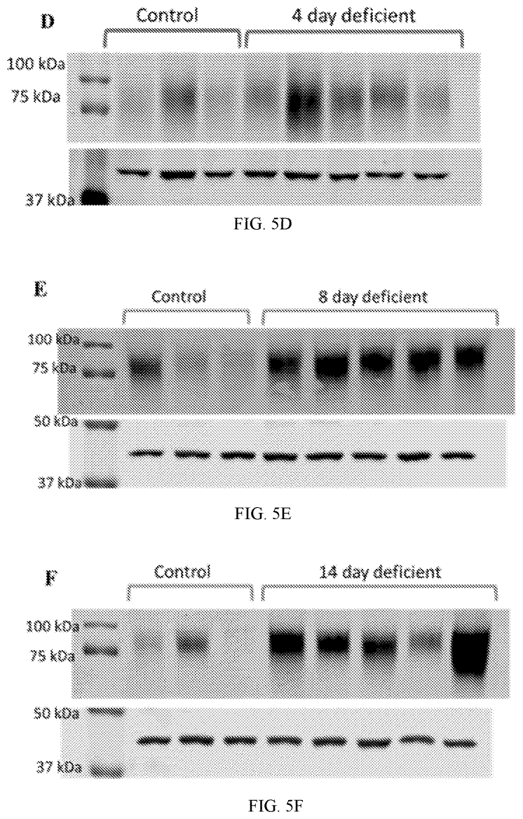

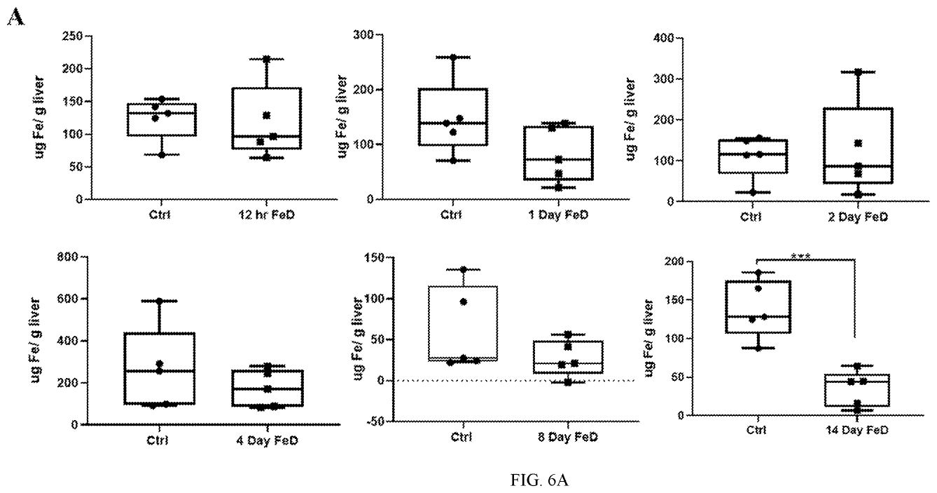

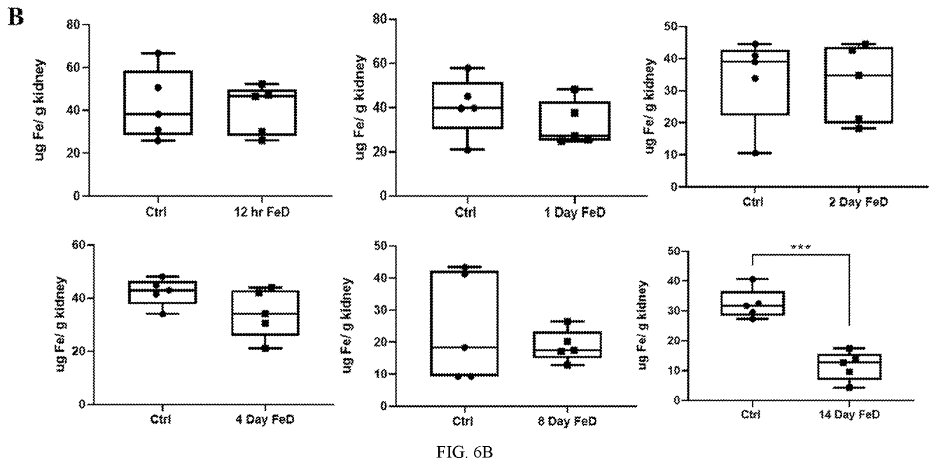

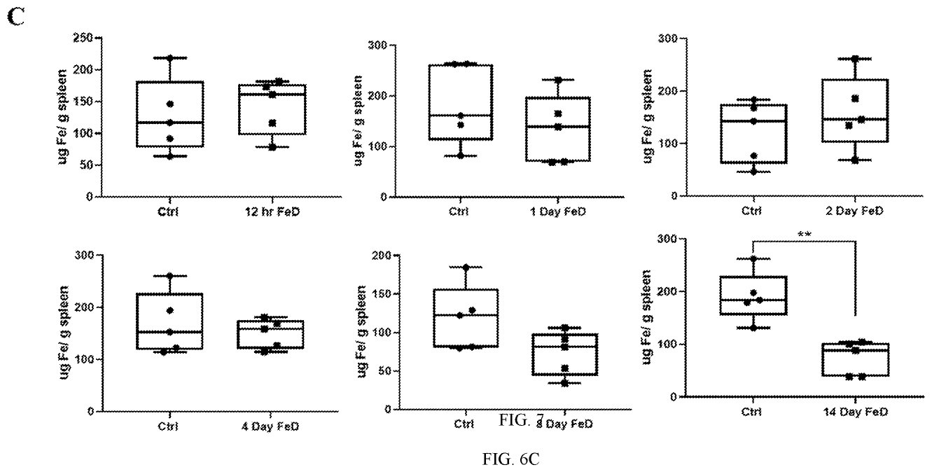

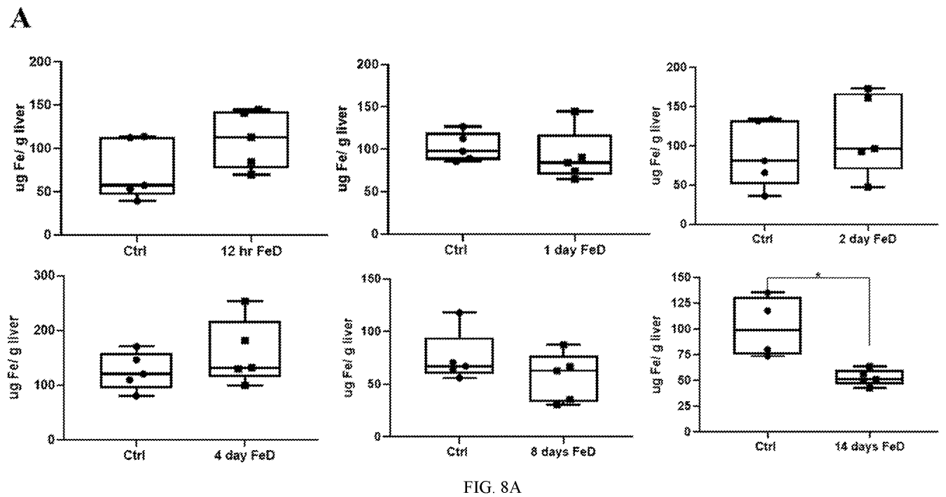

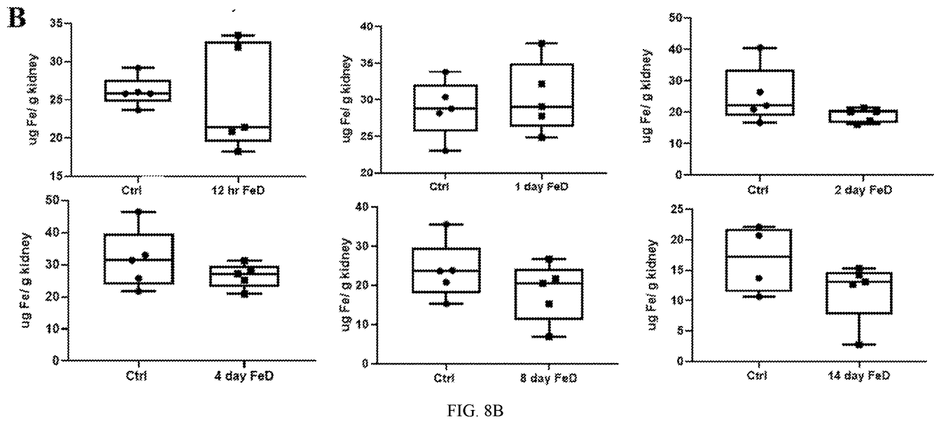

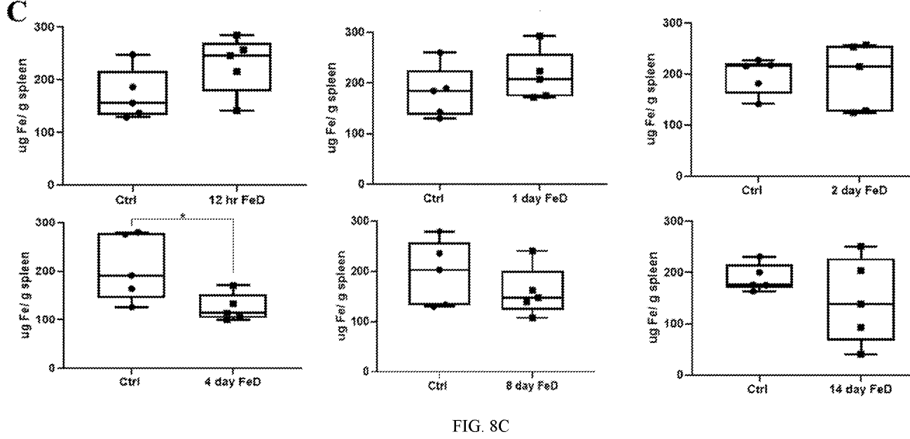

Female adult Swiss Webster mice did not display significant upregulation of DMT1 protein levels until 8 days on the Fe-deficient diet (FIG. 5). This was also consistently observed at 14 days as well. Liver, kidney, and spleen non-heme iron levels were not significantly depleted until 14 days on the Fe-deficient diet (FIG. 6). Male adult Swiss Webster mice did not display significant upregulation of DMT1 protein levels until 14 days on the Fe-deficient diet (FIG. 7). Only the liver spleen non-heme iron levels were not significantly depleted after 14 days on the Fe-deficient diet (FIG. 8).

### Short-Term ⁵⁹Fe Gavage Studies

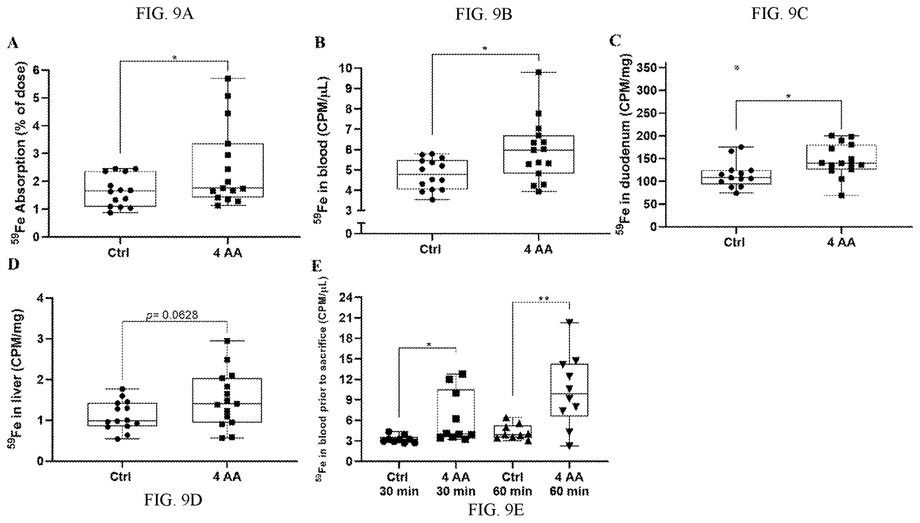

There was a significant increase in ⁵⁹Fe absorption, along with ⁵⁹Fe in the blood and duodenum in male Swiss Webster mice given 4 AA, two hours after given the gavage (FIG. 9). These mice did not have perturbed iron status, as they were fed a chow diet. Activity of ⁵⁹Fe in the liver was increased but did not achieve statistical significance (p=0.0628). Prior to sacrifice, ⁵⁹Fe activity in the blood was significantly higher in the 4 AA group at 30 minutes (p<0.05) and 60 minutes (p<0.01).

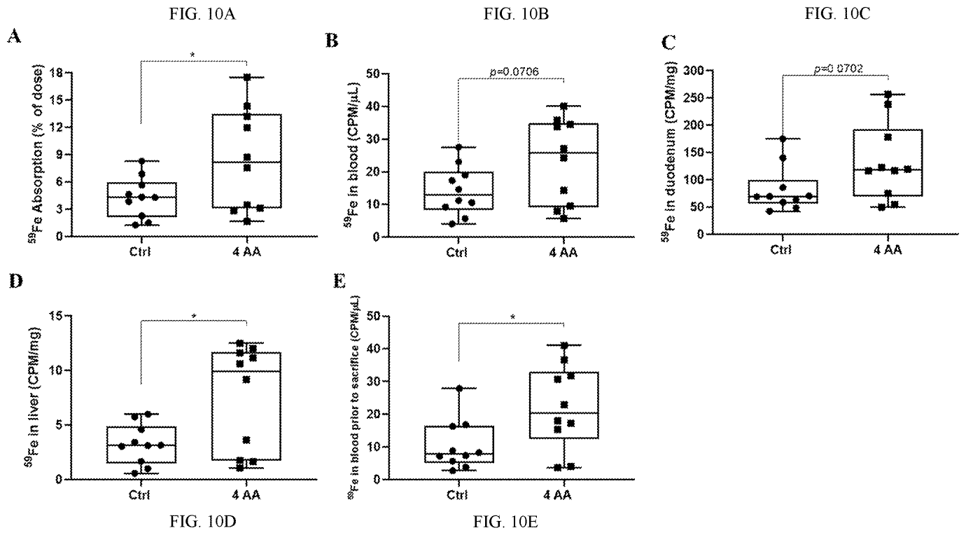

The next experiment was performed where blood was taken 30 minutes after gavage and the mice were sacrificed one hour after gavage. This resulted in a significant increase in ⁵⁹Fe absorption (p<0.05) and ⁵⁹Fe activity in the liver (p<0.05) and blood at 30 minutes (p<0.05) in the 4 AA group compared to controls (FIG. 10). ⁵⁹Fe activity in the duodenum and blood at sacrifice were increased but not did achieve statistical significance in the 4 AA group compared to controls (p=0.0702 and p=0.0706, respectively) (FIG. 10).

### Iron Depletion-Repletion Study

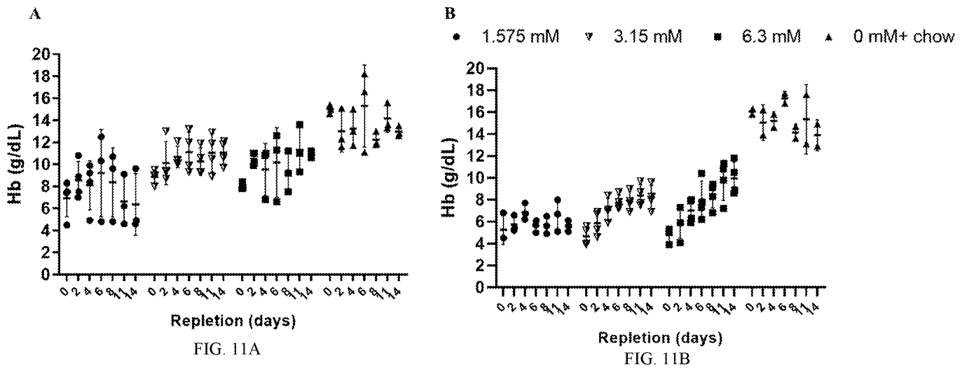

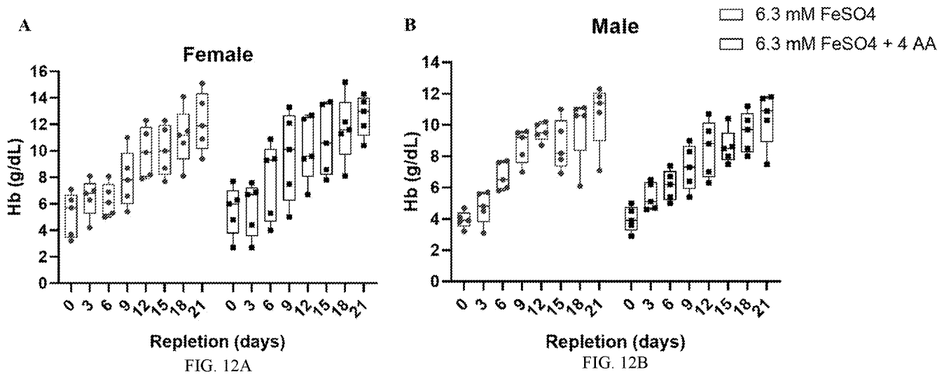

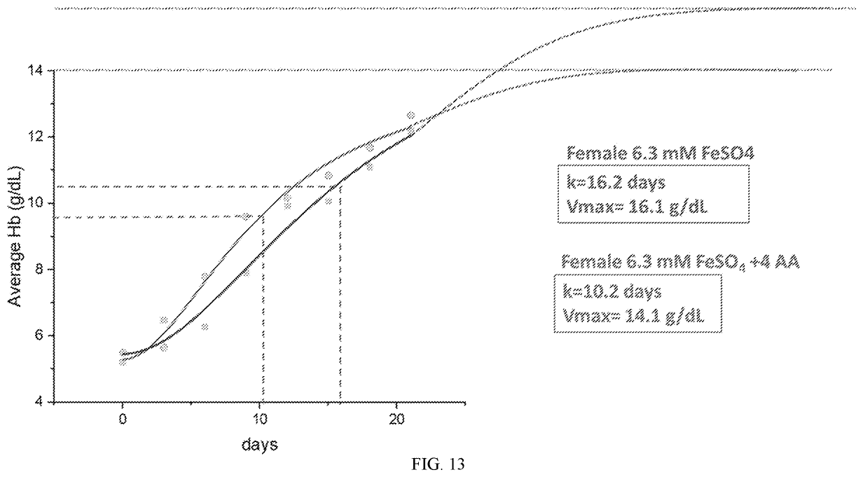

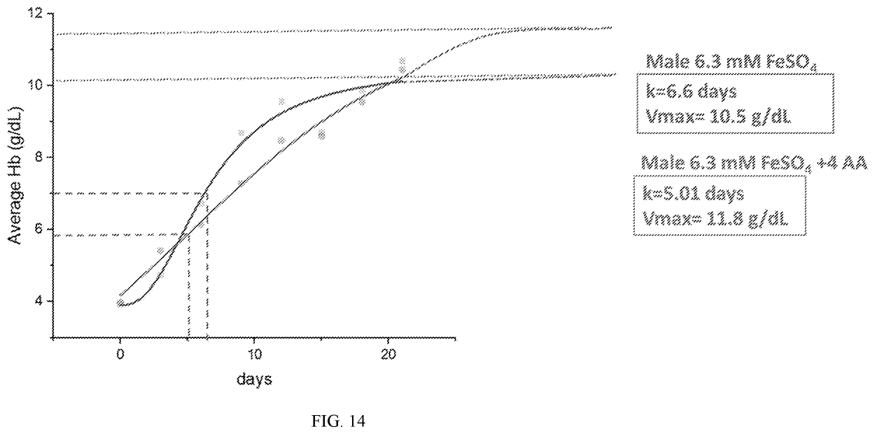

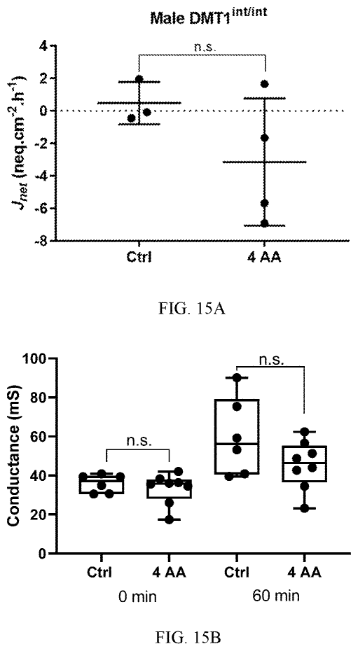

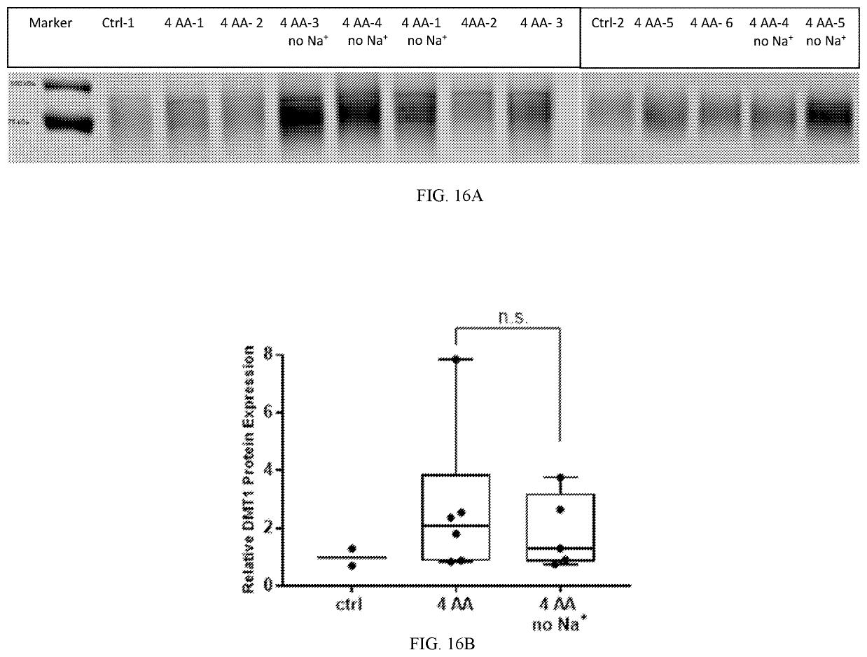

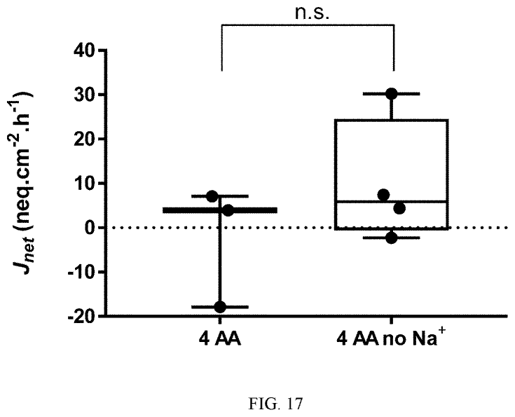

An Fe depletion-repletion study was then performed based on the Fe requirement of a rodent (described previously in the methods). A steady increase was observed in males at 6.3 mM FeSO₄, therefore this concentration was used in the next experiment which included the 4 AA formulation (FIG. 11). Saturation kinetic analysis indicated that female mice achieved half maximal Hb (K_{0.5}) at 16.25 days with control formulation and a Vₘₐₓ of 16.1 g/dL (FIG. 13). Female mice achieved half maximal Hb (K_{0.5}) at 10.2 days with 4 AA formulation and a Vₘₐₓ of 14.1 g/dL. Male mice achieved half maximal Hb (K_{0.5}) at 6.6 days with control formulation and a Vₘₐₓ of 10.5 g/dL (FIG. 14). Male mice achieved half maximal Hb (K_{0.5}) at 5.01 days with 4 AA formulation and a Vₘₐₓ of 11.8 g/dL.

### Effects of Sodium on DMT1 Trafficking and ⁵⁹Fe Flux

Next, to determine if Na⁺ in the 4 AA buffer contributed to the increase in Fe flux, a formulation was created that replaced all compounds containing Na⁺ with NMDG and Hepes buffer. Loop studies were performed with 4 AA formulations with and without Na⁺, and DMT1 protein levels were assessed. The 4 AA average relative DMT1 protein expression was 2.72±2.61 arbitrary units and the 4 AA without Na⁺ average relative DMT1 expression was 1.88±1.29 arbitrary units (FIG. 13). Although not statistically significant, the average DMT1 relative expression is greater when Na⁺ is present. One interpretation of this finding suggests that Na⁺ may be aiding AA apical entry via Na⁺-coupled AA transporters, which may then allow AAs to assist in DMT1 trafficking to BBM. Flux results in FIG. 14 do not show a significant difference among 4 AA with and without Na⁺; however, this will be repeated to clearly determine if there is an effect of Na⁺. Since these flux experiments were performed with a longer incubation time in buffer before the addition of the isotope, some of the tissues were no longer viable at the conclusion of the experiment (due to very high conductance values). Therefore, repeating these experiments with a shorter incubation time is necessary to draw meaningful conclusions regarding the effects of Na⁺ on Fe flux.

### Example 2

### Select Amino Acids Decrease Iron Absorption and can be Utilized as a Therapeutic Approach to Treat Hereditary Hemochromatosis

### Background

The initial 20 AA screen showed clear off-trafficking of DMT1 on the duodenal BBM in the presence of five individual AAs. DMT1 is a therapeutic target for treating hereditary hemochromatosis, in which iron absorption is dysregulated and overactive. By administering a daily dose of the 5 AA formulation, iron loading can be mitigated.

### Methods

### Formulation Design

A formulation was created which included the five AAs that resulted in the lowest expression of DMT1 relative to the control from loop studies (Table 5). The salts, pH of 6.5, and buffering capacity of this solution have been optimized for Fe flux studies by the present inventors. This formulation was utilized for subsequent ex vivo and in vivo studies, as described below.

### Ussing Chamber Flux Studies

Iron flux studies with control and 5 AA formulations (pH 6.5, 1.5 mM FeSO₄) that displayed DMT1 trafficking off the BBM from loop results were performed on male Swiss-Webster mice in the Ussing chamber. Studies will be carried out with individual AAs and pairs of AAs from the formulation. Further modifications to the formulation will be made as necessary. The flux study experimental design is similar to the methods described above in Example 1.

### 5 AA Gavage Study in Weaning Hamp KO and WT Mice

At three weeks of age, Hamp' and Hamp^{-/-} mice were given a daily, evening gavage of control formulation or 5 AA formulation for three weeks. They were given ad libidum access to chow diet and water. Each day a 2-hour fast began at 4:00 pm, gavage occurred at 6:00 pm and food was given back 30 minutes later. After the three-week gavage period, the mice were sacrificed, and blood and tissues were collected for analysis. Serum ferritin (Abcam) was measured by ELISA along with tissue and serum non-heme Fe content. This experimental design was repeated in male Hamp^{-/-} mice with three different diets: chow (200 ppm Fe), control (50 ppm Fe), and low Fe diet (15 ppm Fe) to determine the influence of diet on the outcome.

### Short-Term ⁵⁹Fe Gavage Study

Eight- to ten-week-old Hamp KO mice were maintained on chow diet until five days prior to the experiment, at which point they were placed on Fe control diet (50 ppm Fe). A two hour fast occurred before 300 μL of control or 5 AA formulations were administered to each mouse by oral gavage. Thirty minutes later, 2.5 μCi ⁵⁹Fe was administered by gavage in the presence of 300 μL of control formulation. After the radioactive dose was gavaged, blood was taken from the tail at 30 and 60 minutes to measure ⁵⁹Fe activity. Mice were sacrificed 2 hours after the ⁵⁹Fe gavage to determine percent of dose absorbed, along with ⁵⁹Fe activity in blood, duodenum, and liver.

### Statistical Analysis

All data was analyzed by using GraphPad Prism 7. Unpaired t-tests for flux data and 1-way ANOVA for conductance data were performed.

### Results

### Off-Trafficking Formulation Design and Ussing Chamber Flux Studies

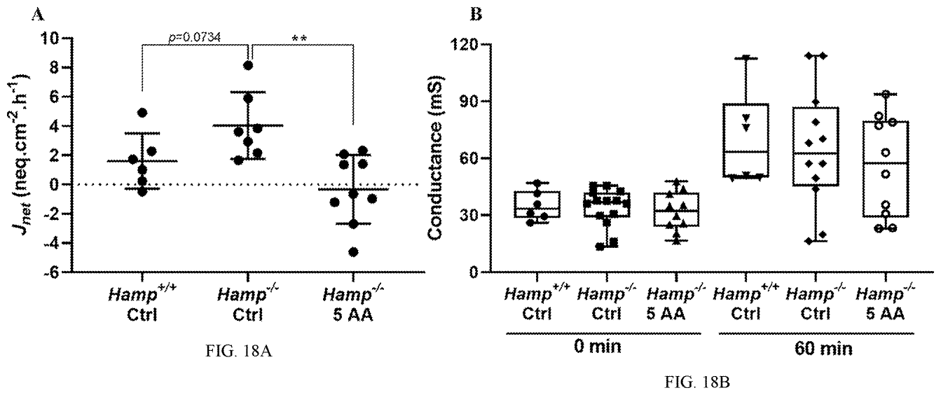

Based off the loop screening of the 20 amino acids, valine, threonine, tyrosine, serine, and lysine were selected as the 5 AA off-trafficking formulation, as they resulted in the lowest relative DMT1 protein expression on the BBM (Table 5). To test this formulation, Ussing chamber ⁵⁹Fe flux studies were conducted in duodenal segments from Hamp^{+/+} and Hamp^{-/-} mice. In the presence of control formulation, duodenal segments from Hamp^{+/+} mice displayed an average ⁵⁹Fe flux of 1.25 neq/cm²·h¹ (FIG. 18A). Duodenal segments from Hamp^{-/-} mice in the presence of control formulation displayed elevated ⁵⁹Fe flux, as expected, with an average of 4.03 neq/cm²·h¹ (FIG. 18A). Duodenal segments from Hamp^{-/-} mice in the presence of 5 AA formulation demonstrated a significant decrease in ⁵⁹Fe flux (p<0.01) compared to Hamp^{-/-} with control formulation (FIG. 18A). The average ⁵⁹Fe flux in the presence of 5 AA was −0.323 neq/cm²·h¹. There was no change in conductance between control and 5 AA formulations at baseline and 60 minutes (FIG. 18B).

TABLE 5
Off-trafficking 5 AA Formulation

| | Molecular weight | Concentration |
|---|---|---|
| Compound | (g/mol) | (mM) |
| NaCl | 58.44 | 86 |
| KCl | 74.55 | 5.2 |
| NaH₂PO₄ | 120 | 7.6 |
| Na₂HPO₄ | 142 | 1.4 |
| MgCl₂ | 95.22 | 1.2 |
| CaCl₂•2 H₂O | 147.03 | 1.2 |
| Na Citrate Monobasic | 214.11 | 25 |
| FeSO₄•7 H₂O | 278.01 | 1.5 |
| Valine | 117.15 | 10 |
| Threonine | 119.12 | 8 |
| Tyrosine | 181.19 | 1.2 |
| Serine | 105.09 | 10 |
| Lysine | 146.190 | 4 |

Formulations were adjusted to pH 6.5 and osmolarity was measured at 294 mOsm.
Note:
concentration of FeSO₄•7 H₂O is only relevant in Ussing chamber flux studies (subsequent gavage studies involving 5 AA formulation do not utilize FeSO₄•7 H₂O).

### 5 AA Gavage Study in Weaning Hamp KO and WT Mice

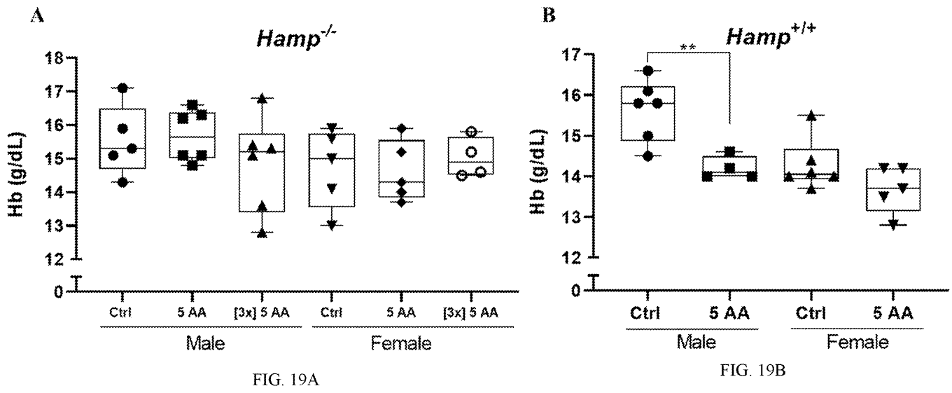

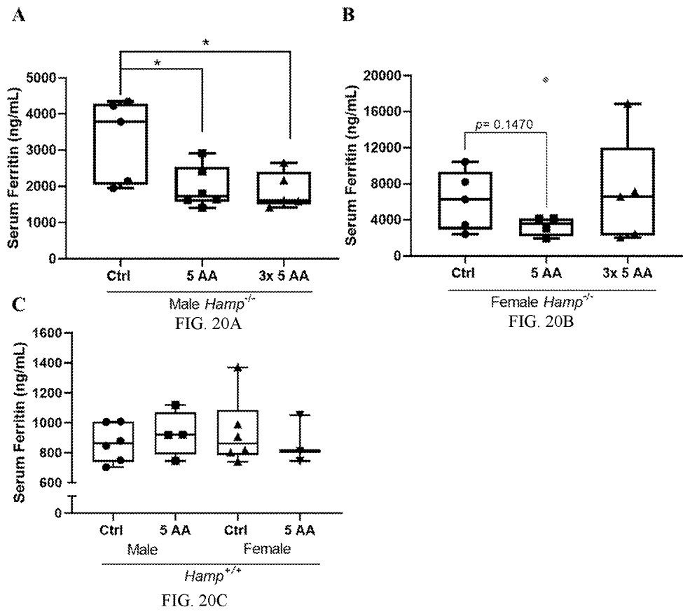

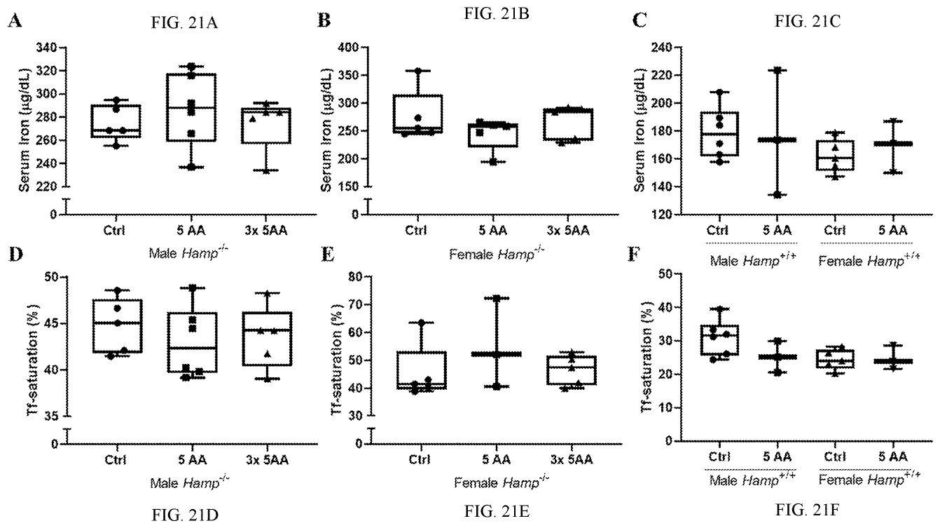

After the three-week daily gavage of control or 5 AA formulations, Hamp KO mice showed no change in Hb (FIG. 19A). The only change in Hb was observed in Hamp WT male mice, which demonstrated a significantly lower Hb when 5 AA was administered (FIG. 19B). The male Hamp KO mice gavaged with 5 AA displayed a significant decrease (p<0.05) in serum ferritin (FIG. 20). The female Hamp KO mice showed a decrease in serum ferritin, but this did not reach statistical significance (p=0.1470). No changes were observed in serum non-heme iron nor transferrin saturation in any of the mice, regardless of sex or genotype (FIG. 21).

### Example 3

### Characterize Fe Flux (Heme and Non-Heme) In Human Intestinal Samples and Test the AA Formulations in These Samples

### Methods and Future Studies

⁵⁹Fe Flux Studies: Human duodenal samples will be resected from Fe-deficient patients undergoing the Whipple procedure and from normal organ donors at Shands Hospital. Samples will be bathed in ice-cold Krebs-Ringer's bicarbonate (KRB) buffer bubbled with 95% O₂ and 5% CO₂ gas mixture and transferred from the operating room to the Cancer Genetic Research Complex immediately. Tissue will be cut along the mesenteric border and pinned flat, placing the mucosa-side down in a sterile dissection dish containing ice-cold sterile KRB bubbled with gas mixture. Tissue will be dissected by removing the muscle layer from the mucosa under dissection microscope. The mucopolysaccharide layer will also be removed. Samples will be mounted onto Ussing chamber cassettes, with an area of 1.13 cm². After balancing the Ussing chamber for at least 30 minutes, tissues will be mounted onto slides and assembled into the Ussing chamber and bathed bilaterally in buffer containing 1.5 mM cold iron sulfate. Experiments will be performed under voltage-clamp conditions. Tissues will equilibrate for approximately 30 minutes and will then be paired for mucosal-to-serosal and serosal-to-mucosal flux based on similar conductance values. Blank measurements will be collected from the cold (non-radioactive) side. ⁵⁹Fe (15 μCi per chamber) will be added to either the mucosal or serosal side. At 15-minute intervals, samples will be collected from the cold side until 60 minutes has been reached. Radioactivity in the samples will be measured using a PerkinElmer gamma counter, so that net Fe flux can be determined. Once Fe kinetics of human duodenal samples have been elucidated, the 4 AA formulation to bring DMT1 to the BBM will be tested to determine whether Fe flux can be enhanced. The AA formulation for off-trafficking will be tested as well, to determine if Fe flux decreases. In addition to the tissue donation, access will be provided to clinical parameters such as medical history, laboratory test results, current treatment information, disease status, and age. Therefore, correlations may be drawn among flux data and hematological parameters.

Additionally, heme Fe flux will be incorporated into this study. With access to radiolabeled heme Fe, both heme and non-heme Fe absorption in human duodenal tissues can be studied.

Statistical Analysis: All data will be analyzed using GraphPad Prism 7 and Origin software.

1. A pharmaceutical formulation for use in treating a disease or disorder associated with iron deficiency in a subject in need thereof,
wherein the pharmaceutical formulation comprises:
a) pharmaceutically active ingredients comprising, consisting essentially of, or consisting of a therapeutically effective amount of each of at least two free amino acids selected from a group of amino acids consisting essentially of aspartic acid, glutamic acid, glutamine, and glycine,
wherein the therapeutically effective amount of each of the at least two free amino acids is sufficient to treat the disease or disorder associated with iron deficiency in the subject; and
b) at least one a pharmaceutically inactive ingredient.

2. A pharmaceutical formulation for use in treating a disease or disorder associated with iron deficiency,
wherein the pharmaceutical formulation comprises:
a) pharmaceutically active ingredients comprising, consisting essentially of, or consisting of a therapeutically effective amount of each of at least two free amino acids selected from a first group of amino acids consisting essentially of aspartic acid, glutamic acid, glutamine, and glycine,
wherein the therapeutically effective amount of each of the at least two free amino acids is sufficient to treat the disease or disorder associated with iron deficiency in the subject;
b) at least one a pharmaceutically inactive ingredient; and
c) optionally further comprising pharmaceutically active ingredients comprising, consisting essentially of, or consisting of a therapeutically effective amount of each of at least one free amino acid selected from a second group of amino acids consisting essentially of cysteine, histidine, and isoleucine,
sufficient to treat the disease or disorder associated with iron deficiency in the subject.

3. The pharmaceutical formulation of claim 1 or claim 2, wherein each of the amino acids is an L-amino acid.

4. The pharmaceutical formulation of any one of claims 1 to 3, wherein the pharmaceutical formulation further comprises water as a pharmaceutically inactive ingredient.

5. The pharmaceutical formulation of any one of claims 1 to 4, wherein the at least one pharmaceutically inactive ingredient comprises a pharmaceutically acceptable carrier, buffer, electrolyte, adjuvant, or excipient.

6. The pharmaceutical formulation of any one of claims 1 to 5, wherein the pharmaceutical formulation is sterile.

7. The pharmaceutical formulation of any one of claims 1 to 6, wherein the pharmaceutical formulation is formulated for administration by an enteral, pulmonary, inhalation, intranasal, or sublingual route.

8. The pharmaceutical formulation of any one of claims 1 to 7, for use as a medicament for the treatment of a disease or disorder associated with iron deficiency.

9. The pharmaceutical formulation of any one of claims 1 to 8,
wherein the pharmaceutically active ingredients consist essentially of or consist of the therapeutically effective amount of each of the at least two free amino acids selected from the group of amino acids consisting essentially of aspartic acid, glutamic acid, glutamine, and glycine; or
the pharmaceutically active ingredients consist essentially of or consist of the therapeutically effective amount of each of the at least two free amino acids selected from the the first group of amino acids consisting essentially of aspartic acid, glutamic acid, glutamine, and glycine and, when present, the therapeutically effective amount of each of the at least one free amino acids selected from the second group of amino acids consisting essentially of cysteine, histidine, and isoleucine.

10. The pharmaceutical formulation of any one of claims 1 to 9, wherein the at least two free amino acids consist essentially of or consist of aspartic acid, glutamic acid, glutamine, and glycine.

11. The pharmaceutical formulation of any one of claims 1 to 9, wherein the at least two free amino acids consist essentially of or consist of aspartic acid and glutamic acid.

12. The pharmaceutical formulation of any one of claims 1 to 9, wherein the at least two free amino acids consist essentially of or consist of aspartic acid, glutamic acid, and glutamine.

13. The pharmaceutical formulation of any one of claims 1 to 9, wherein the at least two free amino acids consist essentially of or consist of aspartic acid and glutamine.

14. The pharmaceutical formulation of any one of claims 1 to 9, wherein the at least two free amino acids consist essentially of or consist of aspartic acid, glutamine, and glycine.

15. The pharmaceutical formulation of any one of claims 1 to 9, wherein the at least two free amino acids consist essentially of or consist of aspartic acid and glycine.

16. The pharmaceutical formulation of any one of claims 1 to 9, wherein the at least two free amino acids consist essentially of or consist of glutamic acid and glutamine.

17. The pharmaceutical formulation of any one of claims 1 to 9, wherein the at least two free amino acids consist essentially of or consist of glutamic acid, glutamine, and glycine.

18. The pharmaceutical formulation of any one of claims 1 to 9, wherein the at least two free amino acids consist essentially of or consist of glutamic acid and glycine.

19. The pharmaceutical formulation of any one of claims 1 to 9, wherein the at least two free amino acids consist essentially of or consist of glutamine and glycine.

20. The pharmaceutical formulation of any one of claims 1 to 9, wherein the at least two free amino acids consist essentially of or consist of aspartic acid, glutamic acid, and glycine.

21. The pharmaceutical formulation of any one of claims 1-20, wherein a concentration of each of the amino acids present ranges from 0.1 mM to 12 mM or 0.5 mM to 12 mM.

22. The pharmaceutical formulation of any one of claims 1-21, wherein, when included, a concentration of valine is 10 mM, a concentration of threonine is 8 mM, a concentration of tyrosine is 1.2 mM, a concentration of serine is 10 mM, and a concentration of lysine is 4 mM.

23. The pharmaceutical formulation of any one of claims 1-22, wherein the pH ranges from 5.5 to 8.0 or is about 6.5.

24. The pharmaceutical formulation of any one of claims 1-23, wherein the disease or disorder associated with iron deficiency comprises iron-deficiency anemia (IDA); anemia associated with chronic kidney disease; iron-refractory, iron-deficiency anemia (IRIDA); anemia associated with inflammation; anemia associated with pregnancy; anemia associated with excessive menstrual blood loss; anemia associated with dietary iron insufficiency; anemia associated with intestinal infections, or anemia associated with inflammatory bowel diseases.

25. The pharmaceutical formulation of claim 24, wherein the anemia comprises iron-deficiency anemia (IDA).

26. A method for treating a disease or disorder associated with iron deficiency in a subject in need thereof, the method comprising:
administering a pharmaceutical composition to the subject in need thereof,
wherein the pharmaceutical composition comprises:
a) pharmaceutically active ingredients comprising, consisting essentially of, or consisting of a therapeutically effective amount of each of at least two free amino acids selected from a group of amino acids consisting essentially of aspartic acid, glutamic acid, glutamine, and glycine, wherein the therapeutically effective amount of each of the at least two free amino acids is sufficient to treat the disease or disorder associated with iron deficiency in the subject; and
b) at least one a pharmaceutically inactive ingredient.

27. A method for treating a disease or disorder associated with iron deficiency in a subject in need thereof, the method comprising:
administering a pharmaceutical composition to the subject in need thereof,
wherein the pharmaceutical composition comprises:
a) pharmaceutically active ingredients comprising, consisting essentially of, or consisting of a therapeutically effective amount of each of at least two free amino acids selected from a first group of amino acids consisting essentially of aspartic acid, glutamic acid, glutamine, and glycine,
wherein the therapeutically effective amount of each of the at least two free amino acids is sufficient to treat the disease or disorder associated with iron deficiency in the subject;
b) at least one a pharmaceutically inactive ingredient; and
c) optionally further comprising pharmaceutically active ingredients comprising, consisting essentially of, or consisting of a therapeutically effective amount of each of at least one free amino acid selected from a second group of amino acids consisting essentially of cysteine, histidine, and isoleucine,
wherein the therapeutically effective amount of each of the at least one free amino acids is sufficient to treat the disease or disorder associated with iron deficiency in the subject.

28. The method of claim 26 or claim 27, wherein each of the amino acids is an L-amino acid.

29. The method of any one of claims 26-28, wherein the pharmaceutical formulation further comprises water as a pharmaceutically inactive ingredient.

30. The method of any one of claims 26-29, wherein the at least one pharmaceutically inactive ingredient comprises a pharmaceutically acceptable carrier, buffer, electrolyte, adjuvant, or excipient.

31. The method of any one of claims 26-30, wherein the pharmaceutical formulation is sterile.

32. The method of any one of claims 26-31, wherein the pharmaceutical formulation is formulated for administration by an enteral, pulmonary, inhalation, intranasal, or sublingual route.

33. The method of any one of claims 26-32, wherein
the pharmaceutically active ingredients consist essentially of or consist of the therapeutically effective amount of each of the at least two free amino acids selected from the group of amino acids consisting essentially of aspartic acid, glutamic acid, glutamine, and glycine; or
the pharmaceutically active ingredients consist essentially of or consist of the therapeutically effective amount of each of the at least two free amino acids selected from the first group of amino acids consisting essentially of aspartic acid, glutamic acid, glutamine, and glycine and, when present, the therapeutically effective amount of each of the at least one free amino acids selected from the second group of amino acids consisting essentially of cysteine, histidine, and isoleucine.

34. The method of any one of claims 26-33, wherein the at least two free amino acids consist essentially of or consist of aspartic acid, glutamic acid, glutamine, and glycine.

35. The method of any one of claims 26-33, wherein the at least two free amino acids consist essentially of or consist of aspartic acid and glutamic acid.

36. The method of any one of claims 26-33, wherein the at least two free amino acids consist essentially of or consist of aspartic acid, glutamic acid, and glutamine.

37. The method of any one of claims 26-33, wherein the at least two free amino acids consist essentially of or consist of aspartic acid and glutamine.

38. The method of any one of claims 26-33, wherein the at least two free amino acids consist essentially of or consist of aspartic acid, glutamine, and glycine.

39. The method of any one of claims 26-33, wherein the at least two free amino acids consist essentially of or consist of aspartic acid and glycine.

40. The method of any one of claims 26-33, wherein the at least two free amino acids consist essentially of or consist of glutamic acid and glutamine.

41. The method of any one of claims 26-33, wherein the at least two free amino acids consist essentially of or consist of glutamic acid, glutamine, and glycine.

42. The method of any one of claims 26-33, wherein the at least two free amino acids consist essentially of or consist of glutamic acid and glycine.

43. The method of any one of claims 26-33, wherein the at least two free amino acids consist essentially of or consist of glutamine and glycine.

44. The method of any one of claims 26-33, wherein the at least two free amino acids consist essentially of or consist of aspartic acid, glutamic acid, and glycine.

45. The method of any one of claims 26-44, wherein a concentration of each of the amino acids present ranges from 0.1 mM to 12 mM or 0.5 mM to 12 mM.

46. The method of any one of claims 26-45, wherein, when included, a concentration of valine is 10 mM, a concentration of threonine is 8 mM, a concentration of tyrosine is 1.2 mM, a concentration of serine is 10 mM, and a concentration of lysine is 4 mM.

47. The method of any one of claims 26-46, wherein the pH of the pharmaceutical composition ranges from 5.5 to 8.0.

48. The method of any one of claims 26-47, wherein the pH is about 6.5.

49. The method of any one of claims 26-48, wherein the disease or disorder associated with iron deficiency comprises iron-deficiency anemia (IDA); anemia associated with chronic kidney disease; iron-refractory, iron-deficiency anemia (IRIDA); anemia associated with inflammation; anemia associated with pregnancy; anemia associated with excessive menstrual blood loss; anemia associated with dietary iron insufficiency; anemia associated with intestinal infections, or anemia associated with inflammatory bowel diseases.

50. The pharmaceutical formulation of claim 49, wherein the anemia comprises iron-deficiency anemia (IDA).
